(19) United States
(12) Patent Application Publication
(10) Pub. No.: US 2003/0087372 A1
(43) Pub. Date: May 8, 2003

(54) Methods of culturing animal cells and polypeptide production in animal cells

(75) Inventors: Neslihan DelaCruz, San Lorenzo, CA (US); Tina Etcheverry, Berkeley, CA (US); Michael Ostland, San Francisco, CA (US)

Correspondence Address: GENENTECH, INC., 1 DNA WAY, SOUTH SAN FRANCISCO, CA, 94080, US

(73) Assignee: GENENTECH, INC.

(21) Appl. No.: 10/167,517

(22) Filed: Jun. 11, 2002

Related U.S. Application Data
(60) Provisional application No. 60/298,013, filed on Jun. 13, 2001.

(51) Int. Cl. C12P 21/02; C12N 5/06

(52) U.S. Cl. 435/69.1; 435/325; 435/320.1

(57) ABSTRACT

A method of improving expression of polypeptides in mammalian cell culture systems is provided. In particular, the invention is directed to methods of culturing mammalian cells under conditions such as, for example, high levels of glucose, where environmental and nutritional conditions are controlled and adjusted so as to improve process robustness and to promote superior expression of polypeptides having desired qualities, e.g., biological activity. The invention also relates to polypeptides, especially antibodies, produced by the methods, and to methods of using such antibodies, and to pharmaceutical compositions containing the polypeptides. The invention also relates to methods of culturing animal cells in a fed batch cell culture to maximize production capacity, viability of the cell culture, and overall enhancement of yield from such cultures.

### FIELD OF THE INVENTION

This invention relates to a method of improving expression of polypeptides in mammalian cell culture systems. In particular, it is directed to methods of culturing mammalian cells under conditions where environmental and nutritional conditions are controlled and adjusted so as to improve process robustness and to promote superior expression of polypeptides having desired qualities, e.g., biological activity. The invention also relates to polypeptides, especially antibodies or fragments thereof, produced by the methods, and to methods of using such antibodies, and to pharmaceutical composition containing the polypeptides. The invention also relates to methods of culturing animal cells in a fed batch cell culture to maximize production capacity, viability of the cell culture, and overall enhancement of yield from use of such cultures.

### BACKGROUND OF THE INVENTION

With the advent of recombinant DNA technology the utility of polypeptides, such as antibodies, which can be produced in recombinant cell cultures has greatly increased. While some recombinant DNA techniques rely on bacterial or yeast cells for the production of polypeptides, production of polypeptides in animal cells (especially mammalian cells) is achieving more significance, particularly in light of the desirability of producing polypeptides that have post-translational modifications needed to ensure full biological activity of the polypeptide. Similarly, cell fusion techniques for preparing hybridomas, which may be cultured to produce monoclonal antibodies (MAbs), are widely used.

Accordingly, techniques have been developed for enhancing cell growth and/or polypeptide production by such genetically modified animal cells. Several groups have looked at the effects of osmolality on cell growth and polypeptide production. See, for example, Stubblefield et al., Cancer Research, 20:1646-1655 (December 1960); Garcia-Perez et al., Journal of Biological Chemistry, 264(28):16815-16821 (1989); Miner et al., Invasion Metastasis, 1:158-174 (1981); GB 2,251,249; EP 481,791; U.S. Pat. No. 5,151,359; U.S. Pat. No. 4,724,206; U.S. Pat. No. 5,122,469; and WO 89/04867. Various osmolality ranges for cell growth or polypeptide production are recommended and, generally, the osmolality of the cell culture medium is increased via the addition of NaCl or amino acids.

Others have discussed the effect of glucose concentration on cell growth and/or polypeptide production in recombinant cell culture. See, for example, Park et al., Biotechnology and Bioengineering, 40:686-696 (1992); Huang et al., Journal of Biotechnology, 18:161-162 (1991); EP 387,840; Reuveny et al., Journal of Immunological Methods, 86:53-59 (1986); Fine et al., In Vitro, 12(10):693-701 (1976); Dircks et al., Exp. Eve Res., 44:951-958 (1987); Mizutani et al., Biochemical and Biophysical Research Communications, 187(2):664-669 (September 1992); Sugiura Biotechnology and Bioengineering, 39:953-959 (1992); WO 88/01643 Graf et al., DECHEMA Biotechnol. Conf., 3:615-618 (1989); Japanese Patent Appln No. JP 1-101882; U.S. Pat. No. 3,926,723; WO 87/00195; and Fleischaker, Jr., Ph.D. Thesis, Massachusetts Institute of Technology, pp. 196-229 (June 1982). Glacken et al., Biotechnol. Bioeng., 28: 1376-1389 (1986) have also studied the effect of glutamine on cell cultures.

U.S. Pat. Nos. 5,856,179 and 6,180,401 describe improved methods of producing polypeptides, including antibodies, in animal cell culture by controlling the osmolality, via control of glucose and glutamine concentration during the process. These patents provide methods of controlling fed batch cell culture conditions for growth of animal cells so as to maintain high cell viability or extend the period of rapid cell growth. The methods can control production of potentially detrimental metabolic waste products, such as lactic acid, during culturing of mammalian cells. The methods also can curtail the increase of osmolality, due to accumulation and neutralization of waste products and subsequent replacement of consumed glucose. Thus, cell viability can be improved by controlling the osmolality and production of waste products, such as lactic acid, during culturing of mammalian cells. These patents describe the use of relatively low amounts of glucose in the culture medium, e.g., less than about 1 g/L.

Current methods of producing antibodies tend to focus on use of adaptive control strategies or utilize glucose and glutamine substitutes to limit byproduct accumulation. Adaptive controlling depends on control algorithms that are difficult to implement and maintain in an active culture environment. Substitution of glucose and glutamine can significantly increase production costs.

Thus, there is a continuing need to provide improved and cost-effective methods of growing animal cells that can produce biologically active polypeptides incorporating desired post-translational modifications.

### SUMMARY OF THE INVENTION

In response to this need there is provided in accordance with the embodiments of the invention improvements to methods for culturing cells to produce polypeptides. Such methods can improve the viability of the cell culture, produce higher yields of desired products, reduce by-product accumulation and/or achieve cost savings through more efficient and productive systems, particularly for large scale production.

In accordance with the invention there is provided a method of producing polypeptide in cell culture comprising

(a) growing in a cell culture medium animal cells that contain an isolated nucleic acid encoding a desired polypeptide, and

(b) culturing the animal cells in a cell culture medium such that they express the polypeptide, wherein glucose is added at the beginning of said culturing or during said culturing to create a glucose concentrations in the medium of greater than 10 g/L during at least some point of the culturing.

In accordance with the invention, there is also provided a monoclonal antibody produced by the method.

There is also provided a method for treating a disorder in a mammal, including administering to the mammal a therapeutically effective amount of the antibody.

There is also provided a monoclonal antibody produced by the method of the invention or a pharmaceutical composition comprising such a monoclonal antibody for use, for example, in the treatment of a disease or disorder in an animal. Such monoclonal antibodies may be, for example, anti-HER2 (see, for example, WO 0115730), antibody 2C4 (see, for example, WO 0115730 and WO 0100245), anti-VEGF (see, for example, WO 0119987), anti-CD11a (see, for example, WO 9949856 and references cited therein), anti-tissue factor (see, for example, WO0891263), IgG4b (see co-pending application PCT/US01/07501), anti-CD40 (see, for example, WO0075348), anti-CD20 (such as, for example antibody C2B8 described in U.S. Pat. No. 5,736,137 and U.S. Pat. No. 6,171,586), and anti-IgE (such as, for example, antibodies E25 and E26 described, for example, in WO9901556, U.S. Pat. No. 6,172,213, and U.S. Pat. No. 5,994,511). These references are herein incorporated by reference in their entirety with respect to descriptions of the monoclonal antibodies, pharmaceutical preparations of these antibodies, and examples of disorders treatable by administration of the antibodies.

There is also provided a method of growing animal cells in fed batch cell culture, comprising growing in a cell culture medium animal cells that contain a nucleic acid encoding a desired polypeptide, and culturing the animal cells in a cell culture medium, wherein glucose is added at the beginning of said culturing or during said culturing to create a glucose concentration during at least some point of said culturing of greater than 10 g/L. Optionally, according to the invention, glucose is added at the beginning of said culturing or during said culturing in one or more increments such that the total amount of glucose added in said culturing is greater than 10 g/L.

In the first aspect of the invention there is provided a method of producing a polypeptide in a cell culture that includes growing in a cell culture medium animal cells that include an isolated nucleic acid encoding a desired polypeptide, and culturing in a production phase the animal cells in a cell culture medium such that the cells express the polypeptide. Glucose is added to the medium at the beginning of culturing or during culturing to create a glucose concentration in the medium of greater than 10 g/L during at least some point of culturing. The method can include an inoculum growth phase wherein the animal cells are expanded to provide an inoculum for the culturing.

In embodiments of this aspect of the invention, the glucose concentration at some point of culturing is at least about 12 g/L up to about 40 g/L, for example, about 12, about 13, about 14, about 15, about 16, about 17, about 18, about 19, or about 20 g/L. The total amount of glucose added at the beginning and during culturing can range from about 10 g/L to and including about 40 g/L, e.g., about 15 to and including about 30 g/L. The glucose can be added in two or more stages during culturing, either alone or as part of a batch feed. Glucose concentration is preferably maintained throughout the culturing to be about 2 g/L. In certain embodiments, the culture medium comprises about 1 to about 12 g/L of glucose, and during or at the beginning of said culturing, at total of greater than 10 g/L of glucose is added to the culture medium.

In other embodiments of the invention, at the start of culturing, the cell culture medium has an osmolality of about 280 to about 380 mOsm, preferably about 300 to about 350 mOsm and at some point of culturing has an osmolality of about 400 to about 600 mOsm, preferably about 420 to and including 500 mOsm.

In other embodiments of the invention, the culturing in a production phase begins at least 3 hours, preferably at least 12 hours, more preferably at least 24 hours, still more preferably at least 48 hours, more preferably about 72 to about 192 hours after the beginning of the growing phase.

In other embodiments, the concentration of glutamine in the medium during culturing is less than about 5 mM, and preferably no glutamine is added to the medium during culturing. In place of or in addition to glutamine, glutamate can optionally be added to the medium during culturing such that the concentration of glutamate is from about 1 to and including about 10 mM during at least a portion of culturing. A ratio of glutamate concentration to glutamine concentration in the cell culture medium is preferably at least 2:0.5.

In other embodiments of the invention, no butyrate is added during culturing, and the culture medium is maintained at a pH between about 6.5 and about 7.5 during culturing.

In other embodiments, a batch feed comprising cell culture nutrients, comprising without limitation, glucose, is added to the cell culture medium during culturing in one or more increments. A batch feed can be the sole source of glucose or be used to supplement glucose added by other methods, i.e., alone. A batch feed can be added to the cell culture medium during culturing once, twice or more. The batch feed is preferably added at the beginning of culturing in the production phase up to and including about 12 hours from the start production culturing, or more preferably between 12 and 120 hours after the start of production culturing, or between about 24 and 72 hours, and again between about 80 and 120 hours, after the start of culturing in the production phase.

In other embodiments, during culturing the temperature of the medium is reduced by at least 2 degrees C. relative to the temperature employed at the beginning of culturing. For example, the temperature can be reduced from about 35 to 39 degrees C. at the beginning of culturing to about 31 to 35 degrees C. for the remainder of culturing. The temperature can be reduced after about 12 to about 72 hours after the beginning of production phase culturing. For example, the temperature can be reduced about 2, about 3, about 4, or about 5 degrees C., at a time of about 12 hours, about 24 hours, about 36 hours, about 48 hours, or about 56 hours, after the beginning of culturing.

In other embodiments, at the beginning of culturing, the seeding density is about 0.1 to about 0.5% PCV. For example, a seeding density of about 0.2% PCV can be used, and a temperature shift can be performed at least at about 24 to and including 72 hours after beginning culturing, wherein the temperature is reduced from about 37° C. to about 34° C. Also, if the seeding density at the beginning of culturing is about 0.4% PCV, a temperature shift can be performed at least at about 3 hours to and including about 72 hours after the beginning of said culturing, wherein the temperature is reduced from about 37° C. to about 33° C.

In still further embodiments, the method of the invention comprises adding an inoculum of cells to the culture medium at the beginning of culturing. According to the invention, the inoculum volume is one-fifth the volume of the culture medium and the inoculum comprises animal cells at a density of about 0.5 to and including about 2.5 packed cell volume, preferably from about 1 PCV to and including about 2 PCV.

In preferred embodiments, the cell culture medium in both the growing and culturing stages is free of serum, preferably free of animal-derived proteins.

In preferred embodiments, the cells are mammalian cells, preferably Chinese Hamster Ovary cells.

In preferred embodiments, the polypeptide is a monoclonal antibody, for example, anti-HER2, antibody 2C4, anti-VEGF, antibody C2B8, anti-CD11a, anti-tissue factor, anti-CD40, anti-CD20, anti-IgE, antibody E25, or antibody E26.

A second aspect of the invention provides a monoclonal antibody produced by the method described above. Preferably the antibody comprises at least one glycan residue and the total number of glycans in the antibody having zero terminal galactose residues is less than about 80%, more preferably less than about 70%, still more preferably less than about 60%, and most preferably about 55% or less.

A third aspect of the invention provides a method for treating a disorder in a mammal, that includes administering to the mammal a therapeutically effective amount of the antibody described above.

A fourth aspect of the invention provides a pharmaceutical composition containing a monoclonal antibody as described above, for example, an anti-HER2, antibody 2C4, anti-VEGF, antibody C2B8, anti-CD11a, anti-tissue factor, IgG4b, anti-CD40, anti-CD20, anti-IgE, E25, or E26.

A fifth aspect of the invention provides a method of growing animal cells in fed batch cell culture, including growing animal cells in a cell culture medium, and culturing the animal cells in the cell culture medium in a polypeptide production phase, wherein glucose is added at the beginning of culturing or during said culturing to create a glucose concentration during culturing of greater than 10 g/L.

In embodiments of this fifth aspect of the invention, the glucose concentration at some point of culturing is at least about 12 g/L up to about 40 g/L, for example, about 12, about 13, about 14, about 15, about 16, about 17, about 18, about 19, or about 20 g/L. The total amount of glucose added at the beginning and during culturing can range from about 10 g/L to and including about 40 g/L, e.g., about 15 to and including about 30 g/L. The glucose can be added in two or more stages during culturing, either alone or as part of a batch feed. Glucose concentration is preferably maintained throughout the culturing to be at least about 2 g/L. In certain embodiments, the culture medium comprises about 1 to about 12 g/L of glucose, and during or at the beginning of said culturing, at total of greater than 10 g/L of glucose is added to the culture medium

In other embodiments of the invention, at the start of culturing, the cell culture medium has an osmolality of about 280 to about 380 mOsm, preferably about 300 to about 350 mOsm and at some point of culturing has an osmolality of about 400 to about 600 mOsm, preferably about 420 to 500 mOsm.

In other embodiments of the invention, the culturing in the production phase begins at least 3 hours, preferably at least 12 hours, and optionally at least 24 hours, at least 48 hours, or at least about 72 to and including about 192 hours after the beginning of the growing phase.

In other embodiments, the concentration of glutamine in the medium during culturing is less than about 5 mM, and preferably no glutamine is added to the medium during culturing. In place of or in addition to glutamine, glutamate can be added to the medium during culturing such that the concentration of glutamate is from about 1 to and including about 10 mM during at least a portion of culturing. A ratio of glutamate concentration to glutamine concentration in the cell culture medium is preferably at least 2:0.5.

In other embodiments of the invention, no butyrate is added during culturing, and the culture medium is maintained at a pH between about 6.5 and about 7.5 during culturing.

In other embodiments, a batch feed is added to the cell culture medium during culturing in one or more increments. A batch feed can be the sole source of glucose or be used to supplement glucose added by other methods, i.e., alone. A batch feed can be added to the cell culture medium during culturing once, twice or more often. The batch feed is preferably added between 3 and 120 hours after the start of culturing, or between about 24 and 72 hours, and again between about 80 and 120 hours, after the start of culturing.

In other embodiments, during culturing the temperature of the medium is reduced by at least 2 degrees C. relative to the temperature employed at the beginning of culturing. For example, the temperature can be reduced from about 35 to 39 degrees C. at the beginning of culturing to about 31 to 35 degrees C. for the remainder of culturing. The temperature can be reduced after about 12 to and including about 72 hours after the beginning of culturing. For example, the temperature can be reduced about 2, about 3, about 4, or about 5 degrees C., at a time of about 12 hours, about 24 hours, about 36 hours, about 48 hours, or about 56 hours, after the beginning of culturing.

In other embodiments, at the beginning of culturing, the seeding density is about 0.1 to about 0.5% PCV. For example, a seeding density of about 0.2% PCV can be used, and a temperature shift can occur at about 24 hours after beginning culturing, wherein the temperature is reduced from about 37° C. to about 34° C. Also, for example, if the seeding density at the beginning of culturing is about 0.4% PCV, a temperature shift can be performed at about 24 hours after the beginning of said culturing, wherein the temperature is reduced from about 37° C. to about 33° C.

In still further embodiments, the method of the invention comprises adding an inoculum of cells to the culture medium at the beginning of culturing. According to the invention, the inoculum volume is one-fifth the volume of the culture medium and the inoculum comprises animal cells at a density of about 0.5 to and including about 2.5 packed cell volume, preferably from about 1 PCV to and including about 2 PCV.

In preferred embodiments, the cell culture medium in both the growing and culturing stages is free of serum, preferably free of animal-derived proteins.

In preferred embodiments, the cells are mammalian cells, preferably Chinese hamster ovary cells.

In preferred embodiments, the polypeptide is a monoclonal antibody, for example, anti-HER2, antibody 2C4, anti-VEGF, antibody C2B8, anti-CD11a, anti-tissue factor, IgG4b, anti-CD40, anti-CD20, anti-IgE, E25, or E26.

Each of the references cited herein is hereby incorporated by reference in its entirety. Further objects, features, and advantages of the invention will become apparent from the detailed description that follows.

### BRIEF DESCRIPTION OF THE DRAWINGS

FIG. 1 depicts a progressive increase in antibody titer with improved culturing conditions according to the invention.

FIG. 2 depicts the increases in ammonium ion level due to the presence of glutamine in the culturing media.

FIG. 3 shows a negative impact on cell viability with increasing glutamine level in the culturing media.

FIG. 4 is graphical representation of the effects of by-product accumulation as a function of temperature, glutamine concentration and glutamate concentration in the culture medium.

### DESCRIPTION OF THE PREFERRED EMBODIMENTS

### Definitions

The processes of the present invention are known in the art as production in a fed batch culture. The phrase “fed batch cell culture,” as used herein refers to a batch culture wherein the animal cells and culture medium are supplied to the culturing vessel initially, and additional culture nutrients are fed, continuously or in discrete increments, to the culture during the culturing process, with or without periodic cell and/or product harvest before termination of culture. Fed batch culture is distinguished from simple “batch culture” in which all components for cell culturing (including the animal cells and all culture nutrients) are supplied to the culturing vessel at the start of the culturing process. Fed batch culture can be further distinguished from perfusion culturing insofar as the supemate is not removed from the culturing vessel during the process (in perfusion culturing, the cells are restrained in the culture by, e.g., filtration, encapsulation, anchoring to microcarriers, etc., and the culture medium is continuously or intermittently introduced and removed from the culturing vessel). However, removal of samples for testing purposes during fed batch cell culture is contemplated.

The process of the current invention can be used to produce polypeptides, including particular antibodies, in any type of animal cells. The term “animal cells” encompasses invertebrate, non-mammalian vertebrate (e.g., avian, reptile and amphibian) and mammalian cells. Examples of invertebrate cells include the following insect cells: Spodoptera frugiperda (caterpillar), Aedes aegypti (mosquito), Aedes albopictus (mosquito), Drosophila melanogaster (fruitfly), and Bombyx mori (See, e.g., Luckow et al., Bio/Technology, 6:47-55 (1988); Miller et al., in Genetic Engineering, Setlow, J. K. et al., eds., Vol. 8 (Plenum Publishing, 1986), pp. 277-279; and Maeda et al., Nature, 315:592-594 (1985)).

In preferred embodiments, the cells are mammalian cells. Examples of mammalian cells include human retinoblasts (PER.C6 (CruCell, Leiden, The Netherlands)); monkey kidney CV1 line transformed by SV40 (COS-7, ATCC CRL 1651); human embryonic kidney line (293 or 293 cells subcloned for growth in suspension culture, Graham et al., J. Gen Virol., 36:59 (1977)); baby hamster kidney cells (BHK, ATCC CCL 10); Chinese hamster ovary cells/-DHFR (CHO, Urlaub and Chasin, Proc. Natl. Acad. Sci. USA, 77:4216 (1980)); mouse sertoli cells (TM4, Mather, Biol. Reprod., 23:243-251 (1980)); monkey kidney cells (CV1 ATCC CCL 70); African green monkey kidney cells (VERO-76, ATCC CRL-1 587); human cervical carcinoma cells (HeLa, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci., 383:44-68 (1982)); MRC 5 cells; FS4 cells; and a human hepatoma line (Hep G2). CHO cells are a preferred cell line for practicing the invention.

The invention is also applicable to hybridoma cells. The term “hybridoma” refers to a hybrid cell line produced by the fusion of an immortal cell line of immunologic origin and an antibody producing cell. The term encompasses progeny of heterohybrid myeloma fusions, which are the result of a fusion with human cells and a murine myeloma cell line subsequently fused with a plasma cell, commonly known as a trioma cell line. Furthermore, the term is meant to include any immortalized hybrid cell line that produces antibodies such as, for example, quadromas (See, e.g., Milstein et al., Nature, 537:3053 (1983)). The hybrid cell lines can be of any species, including human and mouse.

In a most preferred embodiment the mammalian cell is a non-hybridoma mammalian cell, which has been transformed with exogenous isolated nucleic acid encoding a polypeptide of interest, including in especially preferred embodiments, nucleic acids encoding antibodies, antibody fragments, such as ligand-binding fragments, and chimeric antibodies. By “exogenous nucleic acid” or “heterologous nucleic acid” is meant a nucleic acid sequence that is foreign to the cell, or homologous to the cell but in a position within the host cell nucleic acid in which the nucleic acid is ordinarily not found.

An isolated nucleic acid is a nucleic acid molecule that is identified and separated from at least one contaminant nucleic acid molecule with which it is ordinarily associated in the natural source of the polypeptide nucleic acid. An isolated nucleic acid molecule is other than in the form or setting in which it is found in nature. An isolated nucleic acid is preferably a non-chromosomal nucleic acid, i.e. isolated from the chromosomal environment in which it naturally exists. Isolated nucleic acid molecules therefore are distinguished from the nucleic acid molecule as it exists in natural cells. However, an isolated nucleic acid molecule includes a nucleic acid molecule contained in cells that ordinarily express the polypeptide where, for example, the nucleic acid molecule is in a chromosomal location different from that of natural cells.

The expression “osmolality” is a measure of the osmotic pressure of dissolved solute particles in an aqueous solution. The solute particles include both ions and non-ionized molecules. Osmolality is expressed as the concentration of osmotically active particles (i.e., osmoles) dissolved in 1 kg of water (1 mOsm/kg H₂O at 38° C. is equivalent to an osmotic pressure of 19 mm Hg). “Osmolality” refers to the number of solute particles dissolved in 1 liter of solution. Solutes which can be added to the culture medium so as to increase the osmolality thereof include proteins, peptides, amino acids, non-metabolized polymers, vitamins, ions, salts, sugars, metabolites, organic acids, lipids, etc. When used herein, the abbreviation “mOsm” means “milliosmoles/kg H₂O.” According to the invention, glucose is added to a cell culture medium to increase osmolality during the production phase. During the growth phase, the osmolality is preferably from approximately 280 mOsm to and including approximately 380 mOsm, more preferably from about 300 mOsm to and including 350 mOsm. Glucose is added at the beginning or during the production phase to generate an osmolality in the production phase culture medium of from about 400 mOsm to and including about 600 mOsm, more preferably from about 420 mOsm to and including about 500 mOSm.

The word “glucose” refers to either of alpha-D-glucose or beta-D-glucose, separately or in combination. It is noted that alpha- and beta-glucose forms are interconvertible in solution.

The term “glutamine” refers to the amino acid L-glutamine (also known as “Gln” and “Q” by three-letter and single-letter designation, respectively) which is recognized as both an amino acid building block for protein synthesis and as an energy source in cell culture.

Glutamate refers to L-glutamic acid (also known as “Glu” and “E” by three-letter and single-letter designation, respectively).

Butyrate is a straight chain alkanoic acid or salt thereof e.g. sodium butyrate, can be used to enhance protein production.

The terms “amino acids” and “amino acid” refer to all naturally occurring alpha amino acids in both their D and L stereoisomeric forms, and their analogs and derivatives. An analog is defined as a substitution of an atom in the amino acid with a different atom that usually has similar properties. A derivative is defined as an amino acid that has another molecule or atom attached to it. Derivatives would include, for example, acetylation of an amino group, amination of a carboxyl group, or oxidation of the sulfur residues of two cysteine molecules to form cysteine.

As used herein, “polypeptide” refers generally to peptides and proteins having more than about ten amino acids. The polypeptides may be homologous to the host cell, or preferably, may be exogenous, meaning that they are heterologous, i.e., foreign, to the host cell being utilized, such as a human protein produced by a Chinese hamster ovary cell, or a yeast polypeptide produced by a mammalian cell. Preferably, mammalian polypeptides (polypeptides that were originally derived from a mammalian organism) are used, more preferably those which are directly secreted into the medium.

Various polypeptides may be produced according to the invention. Examples of bacterial polypeptides include, e.g., alkaline phosphatase and .beta.-lactamase. Examples of mammalian polypeptides include molecules such as renin, a growth hormone, including human growth hormone; bovine growth hormone; growth hormone releasing factor; parathyroid hormone; thyroid stimulating hormone; lipoproteins; alpha-1-antitrypsin; insulin A-chain; insulin B-chain; proinsulin; follicle stimulating hormone; calcitonin; luteinizing hormone; glucagon; clotting factors such as factor VIIIC, factor IX, tissue factor, and von Willebrands factor; anti-clotting factors such as Protein C; atrial natriuretic factor; lung surfactant; a plasminogen activator, such as urokinase or human urine or tissue-type plasminogen activator (t-PA); bombesin; thrombin; hemopoietic growth factor; tumor necrosis factor-alpha and -beta; enkephalinase; RANTES (regulated on activation normally T-cell expressed and secreted); human macrophage inflammatory protein (MIP-1-alpha); a serum albumin such as human serum albumin; mullerian-inhibiting substance; relaxin A-chain; relaxin B-chain; prorelaxin; mouse gonadotropin-associated peptide; a microbial protein, such as beta-lactamase; DNase; inhibin; activin; vascular endothelial growth factor (VEGF); receptors for hormones or growth factors; integrin; protein A or D; rheumatoid factors; a neurotrophic factor such as bone-derived neurotrophic factor (BDNF), neurotrophin-3, -4, -5, or -6 (NT-3, NT-4, NT-5, or NT-6), or a nerve growth factor such as NGF-.beta.; platelet-derived growth factor (PDGF); fibroblast growth factor such as aFGF and bFGF; epidermal growth factor (EGF); transforming growth factor (TGF) such as TGF-alpha and TGF-beta, including TGF-.beta. 1, TGF-.beta.2, TGF-.beta.3, TGF-.beta.4, or TGF-.beta.5; insulin-like growth factor-I and -II (IGF-I and IGF-II); des(1-3)-IGF-I (brain IGF-I), insulin-like growth factor binding proteins; CD proteins such as CD-3, CD-4, CD-8, and CD-19; erythropoietin; osteoinductive factors; immunotoxins; a bone morphogenetic protein (BMP); an interferon such as interferon-alpha, -beta, and -gamma; colony stimulating factors (CSFs), e.g., M-CSF, GM-CSF, and G-CSF; interleukins (ILs), e.g., IL-1 to IL-10; superoxide dismutase; T-cell receptors; surface membrane proteins; decay accelerating factor; viral antigen such as, for example, a portion of the AIDS envelope; transport proteins; homing receptors; addressing; regulatory proteins; antibodies; and fragments of any of the above-listed polypeptides.

Antibodies are other examples of mammalian polypeptides produced according to the invention. Antibodies are a preferred class of polypeptides that exhibit binding specificity to a specific antigen. Native antibodies are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (V_{H}) followed by a number of constant domains. Each light chain has a variable domain at one end (V_{L}) and a constant domain at its other end; the constant domain of the light chain is aligned with the first constant domain of the heavy chain, and the light chain variable domain is aligned with the variable domain of the heavy chain. Particular amino acid residues are believed to form an interface between the light and heavy chain variable domains.

The term “antibody” is used in the broadest sense and specifically covers monoclonal antibodies (including full length monoclonal antibodies), polyclonal antibodies, multispecific antibodies (e.g., bispecific antibodies), and antibody fragments so long as they exhibit the desired biological activity. Exemplary antibodies are those that are directed against the antigens listed below.

“Antibody fragments” comprise a portion of an intact antibody, generally a portion comprising the antigen binding region or variable region of the intact antibody or the Fc region of an antibody which retains FcR binding capability. Examples of antibody fragments include linear antibodies; single-chain antibody molecules; and multispecific antibodies formed from antibody fragments. The antibody fragments preferably retain at least part of the hinge and optionally the CH1 region of an IgG heavy chain. More preferably, the antibody fragments retain the entire constant region of an IgG heavy chain, and include an IgG light chain.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations that typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al., Nature 256:495 (1975), or may be made by recombinant DNA methods (see, e.g., U.S. Pat. No. 4,816,567). The “monoclonal antibodies” may also be isolated from phage antibody libraries using the techniques described in Clackson et al., Nature 352:624-628 (1991) and Marks et al., J Mol. Biol. 222:581-597 (1991), for example.

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., Proc. Natl. Acad. Sci. USA 81:6851-6855 (1984)).

“Humanized” forms of non-human (e.g., murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, Fv framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992).

An antibody is directed against an antigen of interest. Preferably, the antigen is a biologically important polypeptide and administration of the antibody to a mammal suffering from a disease or disorder can result in a therapeutic benefit in that mammal. However, antibodies directed against nonpolypeptide antigens (such as tumor-associated glycolipid antigens; see U.S. Pat. No. 5,091,178) can also be used.

Where the antigen is a polypeptide, it may be a transmembrane molecule (e.g. receptor) or ligand such as a growth factor. Exemplary antigens include molecules such as renin; a growth hormone, including human growth hormone and bovine growth hormone; growth hormone releasing factor; parathyroid hormone; thyroid stimulating hormone; lipoproteins; alpha-1-antitrypsin; insulin A-chain; insulin B-chain; proinsulin; follicle stimulating hormone; calcitonin; luteinizing hormone; glucagon; clotting factors such as factor VIIIC, factor IX, tissue factor (TF), and von Willebrands factor; anti-clotting factors such as Protein C; atrial natriuretic factor; lung surfactant; a plasminogen activator, such as urokinase or human urine or tissue-type plasminogen activator (t-PA); bombesin; thrombin; hemopoietic growth factor; tumor necrosis factor-alpha and -beta; enkephalinase; RANTES (regulated on activation normally T-cell expressed and secreted); human macrophage inflammatory protein (MIP-1-alpha); a serum albumin such as human serum albumin; Muellerian-inhibiting substance; relaxin A-chain; relaxin B-chain; prorelaxin; mouse gonadotropin-associated peptide; a microbial protein, such as beta-lactamase; DNase; IgE; a cytotoxic T-lymphocyte associated antigen (CTLA), such as CTLA-4; inhibin; activin; vascular endothelial growth factor (VEGF); receptors for hormones or growth factors; protein A or D; rheumatoid factors; a neurotrophic factor such as bone-derived neurotrophic factor (BDNF), neurotrophin-3, -4, -5, or -6 (NT-3, NT-4, NT-5, or NT-6), or a nerve growth factor such as NGF-β; platelet-derived growth factor (PDGF); fibroblast growth factor such as aFGF and bFGF; epidermal growth factor (EGF); transforming growth factor (TGF) such as TGF-alpha and TGF-beta, including TGF-β1, TGF-β2, TGF-β3, TGF-β4, or TGF-β5; insulin-like growth factor-I and -II (IGF-I and IGF-II); des(1-3)-IGF-I (brain IGF-I), insulin-like growth factor binding proteins; CD proteins such as CD3, CD4, CD8, CD18, CD19, CD20, and CD40; erythropoietin; osteoinductive factors; immunotoxins; a bone morphogenetic protein (BMP); an interferon such as interferon-alpha, -beta, and -gamma; colony stimulating factors (CSFs), e.g., M-CSF, GM-CSF, and G-CSF; interleukins (ILs), e.g., IL-1 to IL-10; superoxide dismutase; T-cell receptors; surface membrane proteins; decay accelerating factor; viral antigen such as, for example, a portion of the AIDS envelope; transport proteins; homing receptors; addressins; regulatory proteins; integrins such as CD11a, CD11b, CD11c, CD18, an ICAM, VLA-4 and VCAM; a tumor associated antigen such as HER2, HER3 or HER4 receptor; and fragments of any of the above-listed polypeptides.

Preferred molecular targets for antibodies encompassed by the present invention include CD proteins such as CD3, CD4, CD8, CD18, CD19, CD20, CD34, and CD40; members of the ErbB receptor family such as the EGF receptor, HER2, HER3 or HER4 receptor; cell adhesion molecules such as LFA-1, Mac1, p150.95, VLA-4, ICAM-1, VCAM, α4/β7 integrin, and αv/β3 integrin including either α or β subunits thereof (e.g. anti-CD11a, anti-CD18 or anti-CD11b antibodies); growth factors such as VEGF; tissue factor (TF); alpha interferon (α-IFN); an interleukin, such as IL-8; IgE; blood group antigens; flk2/flt3 receptor; obesity (OB) receptor; mpl receptor; CTLA-4; protein C, and the like.

Soluble antigens or fragments thereof, optionally conjugated to other molecules, can be used as immunogens for generating antibodies. For transmembrane molecules, such as receptors, fragments of these (e.g. the extracellular domain of a receptor) can be used as the immunogen. Alternatively, cells expressing the transmembrane molecule can be used as the immunogen. Such cells can be derived from a natural source (e.g. cancer cell lines) or may be cells which have been transformed by recombinant techniques to express the transmembrane molecule. Other antigens and forms thereof useful for preparing antibodies will be apparent to those in the art.

Multispecific antibodies have binding specificities for at least two different antigens. While such molecules normally will only bind two antigens (i.e. bispecific antibodies, BsAbs), antibodies with additional specificities such as trispecific antibodies are encompassed by this expression when used herein. Examples of BsAbs include those with one arm directed against a tumor cell antigen and the other arm directed against a cytotoxic trigger molecule such as anti-Fc γRI/anti-CD15, anti-p185^{HER2}/FcγRIII (CD16), anti-CD3/anti-malignant B-cell (1D10), anti-CD3/anti-p185^{HER2}, anti-CD3/anti-p97, anti-CD3/anti-renal cell carcinoma, anti-CD3/anti-OVCAR-3, anti-CD3/L-D1 (anti-colon carcinoma), anti-CD3/anti-melanocyte stimulating hormone analog, anti-EGF receptor/anti-CD3, anti-CD3/anti-CAMA1, anti-CD3/anti-CD19, anti-CD3/MoV18, anti-neural cell ahesion molecule (NCAM)/anti-CD3, anti-folate binding protein (FBP)/anti-CD3, anti-pan carcinoma associated antigen (AMOC-31)/anti-CD3; BsAbs with one arm which binds specifically to a tumor antigen and one arm which binds to a toxin such as anti-saporin/anti-Id-1, anti-CD22/anti-saporin, anti-CD7/anti-saporin, anti-CD38/anti-saporin, anti-CEA/anti-ricin A chain, anti-interferon-α (IFN-α)/anti-hybridoma idiotype, anti-CEA/anti-vinca alkaloid; BsAbs for converting enzyme activated prodrugs such as anti-CD30/anti-alkaline phosphatase (which catalyzes conversion of mitomycin phosphate prodrug to mitomycin alcohol); BsAbs which can be used as fibrinolytic agents such as anti-fibrin/anti-tissue plasminogen activator (tPA), anti-fibrin/anti-urokinase-type plasminogen activator (uPA);

BsAbs for targeting immune complexes to cell surface receptors such as anti-low density lipoprotein (LDL)/anti-Fc receptor (e.g. FcγRI, FcγRII or FcγRIII); BsAbs for use in therapy of infectious diseases such as anti-CD3/anti-herpes simplex virus (HSV), anti-T-cell receptor:CD3 complex/anti-influenza, anti-FcγR/anti-HIV; BsAbs for tumor detection in vitro or in vivo such as anti-CEA/anti-EOTUBE, anti-CEA/anti-DPTA, anti-p185^{HER2}/anti-hapten; BsAbs as vaccine adjuvants; and BsAbs as diagnostic tools such as anti-rabbit IgG/anti-ferritin, anti-horse radish peroxidase (HRP)/anti-hornone, anti-somatostatin/anti-substance P, anti-HRP/anti-FITC, anti-CEA/anti-β-galactosidase. Examples of trispecific antibodies include anti-CD3/anti-CD4/anti-CD37, anti-CD3/anti-CD5/anti-CD37 and anti-CD3/anti-CD8/anti-CD37. Bispecific antibodies can be prepared as full length antibodies or antibody fragments (e.g. F(ab′)₂ bispecific antibodies).

Methods for making bispecific antibodies are known in the art. Traditional production of full-length bispecific antibodies is based on the coexpression of two immunoglobulin heavy chain-light chain pairs, where the two chains have different specificities (Millstein et al., Nature, 305:537-539 (1983)). Because of the random assortment of immunoglobulin heavy and light chains, these hybridomas (quadromas) produce a potential mixture of 10 different antibody molecules, of which only one has the correct bispecific structure. Purification of the correct molecule, which is usually done by affinity chromatography steps, is rather cumbersome, and the product yields are low. Similar procedures are disclosed in WO 93/08829, and in Traunecker et al., EMBO J., 10:3655-3659 (1991).

According to a different approach, antibody variable domains with the desired binding specificities (antibody-antigen combining sites) are fused to immunoglobulin constant domain sequences. The fusion preferably is with an immunoglobulin heavy chain constant domain, comprising at least part of the hinge, C_{H}2, and C_{H}3 regions. It is preferred to have the first heavy-chain constant region (C_{H}1) containing the site necessary for light chain binding, present in at least one of the fusions. DNAs encoding the immunoglobulin heavy chain fusions and, if desired, the immunoglobulin light chain, are inserted into separate expression vectors, and are co-transfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

In a preferred embodiment of this approach, the bispecific antibodies are composed of a hybrid immunoglobulin heavy chain with a first binding specificity in one arm, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation (see WO 94/04690). For further details of generating bispecific antibodies see, for example, Suresh et al, Methods in Enzymology, 121:210 (1986). According to another approach, the interface between a pair of antibody molecules can be engineered to maximize the percentage of heterodimers which are recovered from recombinant cell culture (see WO96/27011). The preferred interface comprises at least a part of the C_{H}3 domain of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Such antibodies have, for example, been proposed to target immune system cells to unwanted cells (see U.S. Pat. No. 4,676,980), and for treatment of HIV infection (see WO 91/00360, WO 92/200373, and EP 03089). Heteroconjugate antibodies may be made using any convenient cross-linking methods. Suitable cross-linking agents are well known in the art, and are disclosed in U.S. Pat. No. 4,676,980, along with a number of cross-linking techniques.

Antibodies with more than two valencies are contemplated. For example, trispecific antibodies can be prepared (see Tutt et al. J. Immunol. 147:60 (1991)).

Preferred antibodies produced by the method of the invention include without limitation anti-HER2, antibody 2C4, anti-VEGF, antibody C2B8, antiCD11a, anti-tissue factor, IgG4b, anti-CD40, anti-CD20, anti-IgE, E25, and E26.

The terms “cell culture medium” and “culture medium” refer to a nutrient solution used for growing mammalian cells that typically provides at least one component from one or more of the following categories:

1) an energy source, usually in the form of a carbohydrate such as glucose;

2) all essential amino acids, and preferably, and most commonly, the basic set of twenty amino acids plus cysteine;

3) vitamins and/or other organic compounds required at low concentrations;

4) free fatty acids; and

5) trace elements, where trace elements are defined as inorganic compounds or naturally occurring elements that are typically required at very low concentrations, usually in the micromolar range.

The nutrient solution may optionally be supplemented with one or more components from any of the following categories:

1) hormones and other growth factors as, for example, insulin, transferrin, and epidermal growth factor;

2) salts and buffers as, for example, calcium, magnesium, and phosphate;

3) nucleosides and bases such as, for example, adenosine and thymidine, hypoxanthine; and

4) protein and tissue hydrolysates.

The growth phase refers to the period of exponential growth where cells are generally rapidly dividing, e.g., “growing”. During this phase, cells are cultured for a period of time, usually 1 to 4 days, e.g. 1, 2, 3, or 4 days, and under such conditions that cell growth is optimal. The determination of the growth cycle for the host cell can be determined for the particular host cell by methods known to those skilled in the art.

As used herein, the term “inoculum” refers to a volume of animal cells harvested from growing in a culture medium for addition to a culture medium at the beginning of a production phase. Preferably, the animal cells are “expanded” during the growing, whereby as the cells divide and increase in cell number (i.e., cell density), the cells are transferred to a larger volume of growth medium for continued growth. Preferably, the inoculum has a cell density of from about 0.5 packed cell volume (PCV) to and including about 2.5 PCV, more preferably from about 1 PCV to and including about 2 PCV. Preferably the inoculum volume is one-fifth the volume of the culture medium to be used in the production phase. According to the invention, the animal cells are preferably mammalian cells, more preferably CHO cells.

As used herein, the term “seeding” refers to the addition or inoculation of growing cells into a culture medium at the beginning of the production phase. Further, as used herein, the term “seed train” refers to a continual passaging of cells in volumes of culture medium of about 20 L or less for the maintenance of the cell line.

### Methods of Carrying Out the Invention

The present invention provides benefits of improved cell culture viability and higher production yields of desired polypeptides, such as an antibody expressed from a heterologous nucleic acid within a cell in the culture, through manipulation of discrete factors during the culturing processes based on a greater understanding of the principal effects of and interactions between the various process parameters. In particular, as disclosed herein, manipulation of temperature, particularly in relation to temperature shifts at defined time intervals, media choice, pH shifts at specified intervals, batch feed additions, glutamine/glutamate ratios and glucose concentration all have significant effects on the cell culture process. Moreover, interactions between factors, including between final temperature and the timing of temperature shifts, between media choice and temperature, between seeding density and temperature, concerning pH variance, between glutamine presence, concentrations and ratios to glutamate concentrations, and, in particular, in relation to glucose concentrations, were employed to refine and develop a variety of optimized parameter sets for governing cell culture and production in order to maximize cell culture viability and polypeptide production yield.

Using a stepwise process, the present inventors have developed improved cell culture procedures for producing glycosylated polypeptides, such as monoclonal antibodies or fragments thereof, such as ligand-binding fragments. Preferably, the cells of the cell culture are animal cells, more preferably mammalian cells, and most preferably Chinese hamster ovary cells. Improvements were realized through higher product titers, higher product quality, improved cell culture viability and reduction of undesired by-products within the cell culture environment. According to the invention, product titer and product quality refer to the titer and quality of a polypeptide or protein (such as an antibody or a fragment thereof, such as a ligand-binding fragment thereof) expressed from a heterologous isolated nucleic acid within the cell cultured according to the invention.

A significant number of experiments under varying environmental and nutritional conditions ordinarily must be conducted to develop and optimize a bioprocess. Conventional methods of process development tend to alter one parameter while keeping other parameters constant. This method of process development is expensive, time consuming and to a large extent, gives little information about the mutual interactions of the culture parameters. When interactions exist, it may be difficult to optimize the process with such conventional methods. A better understanding of interactions among the factors and variances in such factors is also important to understand possible implications for the production environment from the alteration of factors to obtain optimal process settings.

Statistical experimental design provides efficient and effective process development (See, for example, Statistics for Experimenters, Box, G.E.P., Hunter, J. S., 1978, Wiley and Sons, New York; and A Guide to Statistics and Data Analysis Using JMP and JMP In® Software, Sall, J. and Lehman, A., SAS Institute, 1996, Duxbury Press). In this approach a response of interest (i.e. product titer, product quality, viability and by-product accumulation) is measured as some factors in the culture medium are changed systematically, while other factors are held constant, within standard experimental variability.

Considerable scientific judgment is needed to decide which factors in a production environment to change and to define the range of possibilities for such changes. The statistical methods noted above were used to identify, measure the effect of, and verify alterations to production factors for which a change over the range of values studied produced a change (or had a principal effect) in average response. The magnitude of the effects of changes to the production environment was estimated and subsequently verified through this process.

Standard errors and P-values respectively express the statistical uncertainty in estimates and the likelihood that one would observe an individual principal effect of similar magnitude under the assumption that the variable did not produce a change in average response. In addition to such individual effects, some statistical designs also allow for an estimation of effects resulting from interactions of selected parameters, thereby quantifying the synergy between two or more variables in their impact on the response.

To develop the improved processes of the present invention, a stepwise full factorial design and analysis approach was employed. Eleven factors affecting cell culture production for animal cell production systems were analyzed, in particular mammalian cell lines such as Chinese hamster ovary cell (“CHO cell”) cultures. Stepwise experiments were used to identify the factors that could be adjusted to yield improvements as noted above. The approach used to design the improved processes of the present invention produced a significant quantity of data supporting the conclusions reached as to effects and ranges in effects on antibody production from changes in the cell culturing factors. Such data were obtained by performing the experiments in small groups and by using the information generated in one experiment to assist in designing subsequent experiments. Use of a single large experiment would not allow one to allocate runs efficiently or generate sufficient data to reinforce the conclusions reached on acceptable variances for the factors studied, since the interactive effects of variables could not have been predicted or measured by such an approach.

Considerable scientific judgement was used to determine the viable and appropriate ranges for such variables. If the ranges used were too small, one might not be able to detect an effect from the change in the factor. If the ranges are too great or disparate, one may move outside of the viable range of verifiable support for the effect of such factors on the process. Furthermore, the range of variation for individual factors is limited to settings that are not be more precise than the ranges that can be employed in a large-scale manufacturing setting. Finally, a stepwise approach allowed for the setting of ranges using a wider variation on the earlier stages of the cell culturing process, and helped in identifying the variables that have main effects at that stage. This permitted the investigation in later stages in the cell culturing process to obtain more detail as to acceptable ranges of the key variables.

### Cell Culture Procedures

Mammalian cell culture procedures useful for practicing the invention are described herein.

1. Cell Culture Growth Phase

An initial step of the process of the invention is a growth phase, wherein batch cell culture conditions are modified to enhance growth of recombinant animal cells, to produce a seed train. The growth phase refers to the period of exponential growth where cells are generally rapidly dividing, e.g. growing. During this phase, cells are cultured for a period of time, usually 1 to 4 days, e.g. 1, 2, 3, or 4 days, and under such conditions that cell growth is optimal. The determination of the growth cycle for the host cell can be determined for the particular host cell by methods known to those skilled in the art.

In the growth phase, the basal culture medium and animal cells are supplied to the culturing vessel in batch. The culture medium is preferably free of serum, e.g. less than about 5%, preferably less than 1%, more preferably 0 to 0.1% serum, and other animal-derived proteins. However, they can be used if desired. In a preferred embodiment of the invention the cell culture medium comprises excess amino acids. The amino acids that are provided in excess may, for example, be selected from Asn, Asp, Gly, Ile, Leu, Lys, Met, Ser, Thr, Trp, Tyr and Val. Preferably, Asn, Asp, Lys, Met, Ser and Trp are provided in excess. For example, amino acids, vitamins, trace elements and other media components at one or two times the ranges specified in European Patent EP 307,247 or U.S. Pat. No. 6,180,401 may be used, which documents are herein incorporated by reference in their entirety.

Alternatively, commercially available media such as Ham's F10 (Sigma), Minimal Essential Medium ([MEM], Sigma), RPMI-1640 (Sigma), and Dulbecco's Modified Eagle's Medium ([DMEM], Sigma) are suitable for culturing the animal cells. In addition, any of the media described in Ham and Wallace, Meth. Enz., 58:44 (1979), Barnes and Sato, Anal.

Biochem., 102:255 (1980), U.S. Pat. Nos. 4,767,704; 4,657,866; 4,927,762; or 4,560,655; WO 90/03430; WO 87/00195; U.S. Pat. No. Re. 30,985; or U.S. Pat. No. 5,122,469, the disclosures of all of which are incorporated herein by reference in their entirety, may be used as culture media for the host cells.

Any of these media may be supplemented as necessary with hormones and/or other growth factors (such as insulin, transferrin, or epidermal growth factor), ions (such as sodium, chloride, calcium, magnesium, and phosphate), buffers (such as HEPES), nucleosides (such as adenosine and thymidine), trace elements (defined as inorganic compounds usually present at final concentrations in the micromolar range), and glucose or an equivalent energy source. Any other necessary supplements may also be included at appropriate concentrations that would be known to those skilled in the art.

The initial media preferably has a glucose concentration in the initial growth phase of about 1 to about 12 g/L, most preferably 6-10 g/L. As discussed below, additional glucose is added during the culturing of the production phase, in at least one increment, and preferably in one, two or three increments, so that a concentration of at least about 10 g/L, preferably at least about 12 g/L, more preferably about 15 to at least about 30 g/L is present during the culturing.

At a particular point in their growth, the cells may form an inoculum to inoculate a culture medium at the start of culturing in the production phase. Alternatively, the production phase may be continuous with the growth phase.

A suitable initial cell seed density for the cell growth phase is in the range 3×10⁵ to 1.5×10⁶ cells/ml, for example. A suitable culturing vessel for cell growth is a pH controlled bioreactor. An autoclavable glass fermenter (sold by Applikon, Foster City, Calif.) or stainless steel fermenter (sold by Biolafitte, Princeton, N.J.) can be used. Other culturing vessels suitable for practicing the invention are well known in the art.

While the cells of the growth phase need not be transformed with exogenous nucleic acid, in the preferred embodiment of the invention, the cell growth phase is followed by a distinct polypeptide, e.g., antibody production phase wherein the cells have been transformed with exogenous nucleic acid encoding the polypeptide of interest. Suitable methods for transformation of the animal cells follow.

2. Transformation of Animal Cells

Methods, vectors, and host cells suitable for adaptation to the synthesis of the polypeptide of interest in recombinant vertebrate cell culture are described in Gething et al., Nature, 293:620-625 (1981); Mantei et al., Nature, 281:40-46 (1979); Levinson et al.; EP 117,060; and EP 117,058. A particularly useful plasmid for mammalian cell culture expression of the polypeptide is pRK5 (EP pub. no. 307,247) or pSVI6B (PCT pub. no. WO 91/08291 published Jun. 13, 1991).

Host cells are transformed with expression or cloning vectors and cultured in nutrient media modified as appropriate for inducing promoters, selecting transformants, or amplifying the genes encoding the desired sequences. For mammalian cells, the calcium phosphate precipitation method of Graham and van der Erb, Virology, 52:456-457 (1978) or the lipofectamine™ (Gibco BRL) Method of Hawley-Nelson, Focus 15:73 (1193) are preferred. General aspects of mammalian cell host system transformations have been described by Axel in U.S. Pat. No. 4,399,216 issued Aug. 16, 1983. For various techniques for transforming mammalian cells, see Keown et al., Methods in Enzymology (1989), Keown et al., Methods in Enzymology, 185:527-537 (1990), and Mansour et al., Nature, 336:348-352 (1988).

The invention also encompasses hybridomas which secrete monoclonal antibodies in cell culture. Monoclonal antibodies are prepared by recovering immune cells (typically spleen cells or lymphocytes from lymph node tissue) from immunized animals and immortalizing the cells in conventional fashion, e.g., by fusion with myeloma cells or by Epstein-Barr (EB)-virus transformation and screening for clones expressing the desired antibody. The hybridoma technique described originally by Kohler and Milstein, Eur. J. Immunol., 6:511 (1976), and also described by Hammerling et al., In: Monoclonal Antibodies and T-Cell Hybridomas, Elsevier, N.Y., pp. 563-681 (1981) has been widely applied to produce hybrid cell lines that secrete high levels of monoclonal antibodies against many specific antigens.

3. Polypeptide Production and the Cell Culturing Phase

The cell growth phase is generally followed by a polypeptide production phase, which is distinct therefrom. The phase is also referred to as a cell-culturing phase. Culturing means the cells are manipulated in a production phase so that they express the desired polypeptide. This phase generally begins at least 3 hours after the beginning of the growth phase, preferably about 12 to about 224 hours, more preferably about 120 to 192 hours after the beginning of the growth phase. The production phase can last, e.g., from 7 to 14 days. During this phase, cell growth has generally plateaued, e.g., logarithmic cell growth has ended and protein production is primary. During this period, as discussed below, the medium is supplemented with glucose and optionally other components. In a preferred embodiment, the production phase may be carried out in a different culturing vessel from the cell growth phase. However, the same vessel can be employed for each step.

The production phase involves inoculating the cultured animal cells of the growth phase at a cell seed density of generally at least about 0.5×10⁵ cells/mL, preferably in the range 1.0-3.0×10⁶ cells/mL. The same media as used in the initial growth steps can be used. However, batch additions of glucose and optionally other components are made.

In order to achieve a culture medium having the desired glucose concentration, a cell culture media providing glucose can be used. Also, the culture medium preferably contains an excess of amino acids in order to provide additional cell nutrients. Optionally, the concentration(s) of other constituents in the culture medium can be adjusted in order to reach the desired osmolality. In the growth phase, the osmolality is preferably from approximately 280 mOsm to and including approximately 380 mOsm, more preferably from about 300 mOsm to and including 350 mOsm. Glucose is added at the beginning or during the production phase to generate an osmolality in the production phase culture medium of from about 400 mOsm to and including about 600 mOsm, more preferably from about 420 mOsm to and including about 500 mOSm.

The various embodiments of the invention involve alteration of the culture medium or culture environment during the culturing of the animal cells. The present disclosure uniquely provides that the individual variation of certain culturing factors produced statistically significant advantages. The present disclosure further uniquely provides that combinations of these individual variables can be employed to yield improvements in one or more properties of a polypeptide product of the culturing process.

A first discovery that led to the invention demonstrated a significant positive affect on production by the use of high concentrations of glucose in the culturing phase. The high glucose concentrations can improve yield of product and viability of the cell culture. According to the invention, the term “product” refers to, for example, a polypeptide or protein (such as an antibody or ligand-binding fragment thereof) expressed from a isolated nucleic acid in the cell. Preferably, the isolated nucleic acid is heterologous to the cell into which it is introduced and/or the isolated nucleic acid is non-chromosomal. As discussed infra, it was particularly surprising to find that increasing glucose concentrations during the culturing process, or the addition of high amounts of glucose (e.g., greater than about 10 g/L) during culturing in the production phase, especially in concentrations or additions higher than heretofore known in the art, was an important factor in improving production yields. The high glucose levels provide these benefits by enabling the cell culture to be maintained in a medium with a high osmolality. By maintaining high osmolality through the use this process, it is possible to reduce or eliminate use of other agents (e.g., sodium chloride) ordinarily used to maintain said higher media osmolality without incurring negative effects such as build-up of undesired by-products. According to the invention, glucose is added during the culturing phase to increase the osmolality of the culture medium. In the growth phase, the osmolality is preferably from approximately 280 mOsm to and including approximately 380 mOsm, more preferably from about 300 mOsm to and including 350 mOsm. Glucose is added at the beginning or during the production phase to generate a high osmolality in the production phase culture medium of from about 400 mOsm to and including about 600 mOsm, more preferably from about 420 mOsm to and including about 500 mOsm.

A second significant culturing discovery concerned use of increased ratio of glutamate to glutamine in the culturing medium. This second discovery enables exploitation of the higher glucose concentrations to maintain desired osmolality. The reduced glutamine content, alone or in relation to glutamate concentration, is particularly useful in reducing creation of unwanted by-products.

A third discovery of significant culturing factors concerns temperature shifts employed during the culturing process of developing a viable cell culture, where temperature shifts in combination with high glucose levels and glutamine and glutamate ratios enhance polypeptide production.

A fourth discovery involved use of one or more additions of concentrated nutrient mixtures (“batch feed”) to an existing viable cell culture during the early and mid-production culturing phases. In particular, addition of one or two batch feed mixtures to the production vessel containing the cells, maintained cell viability and productivity. According to the invention, a preferred batch feed had initial media components in concentrated form (e.g., 4-fold concentrated), which when added to the production vessel restored between 30-40% of the original basal amount of the media components (e.g., 30% of original peptone, greater than 100% glucose, and 100% trace elements). In certain embodiments, selected components will be omitted or their concentrations (e.g., glutamine) will be reduced in the batch feed to ensure that final concentrations of such components in the production vessel are within desired ranges.

The production phase is preferably carried out in the presence of a concentration of glucose controlled; throughout the culturing to be within a range between about 2 to about 40 g/L. It is preferred that during the production phase, from 10 to 40 g/L, preferably from 12-30, more preferably from 15-25 total g/L of total glucose are added. This amount can be in one or more increments, for example, one, two, or three increments. The glucose can be added as pure glucose or as part of e.g., a batch feed. The glucose is added such that the concentration of glucose during at least some portion of the culturing is greater than about 10 g/L, more preferably greater than about 12, most preferably greater than about 15, up to and including about 40 g/L glucose. For example, the amount of glucose measured during this stage can be at least about 12, at least about 13, at least about 14, at least about 15, at least about 16, at least about 17, at least about 18, at least about 19 or more g/L. This glucose concentration will be reduced as it is consumed by the cells. Preferably the amount of glucose is not allowed to fall below about 2 g/L. At least one 10-40 g/L feed of glucose is added at the beginning of the production phase, e.g., after the cell mass has grown sufficiently so that the glucose will not impede the growth phase of the culture, or at any time during the production phase, e.g. from about 12 to about 96 hours after the start of the culturing in production phase. For example, about 10 to about 20 g/L glucose can be added at the beginning of the culturing phase, and optionally at least about 2 to and including at least about 10 g/L can be added in one or more increments about 24 hours after the beginning of the culturing phase.

The high glucose concentrations during the production phase (equivalent to the culturing phase) for a cell culture affords a principal benefit of increasing osmolality of the cell culture medium without the problems associated with inducing high osmolality through use of other agents such as sodium chloride or glutamine. Culturing in a highly osmotic medium enables the desirable result of higher levels of specific productivity of the cell culture.

Intermittent off-line sampling of the culture medium can be carried out. The glucose concentration of the culture medium can then be modified, manually or automatically, by the modulation of a glucose feed solution as required.

Preferably, the glutamine concentration is also controlled throughout the culturing to be in a range of 0 to 15 mM, more preferably 0.5 to 5 mM. This can be done by adding from 0 to 15 mM of glutamine during the culturing. Because the present process uses a low amount of glutamine, the amount of glucose can be increased to amounts greater than in previously believed possible (see, e.g., U.S. Pat. No. 5,856,179), without adversely affecting product quality or adversely increasing by-product accumulation. It is often preferred not to added any glutamate during culturing.

In place of or in addition to glutamine, glutamate can be added. For example, from 0.5 to about 15 mM, e.g. from about 1 to about 10 mM of glutamate can be added during culturing. A ratio of glutamine to glutamate of from 0:2 to 2:0, preferably less than about 0.5:2 can be used.

In at least one embodiment of the invention, the glutamine in the culture medium is lowered or replaced by glutamate. Low glutamine concentrations (e.g., 7.5 mM or less) are desirable in cell culture media because glutamine-leads to the production of waste products such as ammonium and lactic acid which can have directly negative effects on cell growth and/or production. Reduction of waste-products tends to limit the osmolality rise of the culture. Lower osmolality is not beneficial to specific productivity but this adverse effect is partially overcome in the present process by using considerably greater amounts of glucose then conventionally used.

The medium can optionally contain butyrate. Butyrate is known to enhance protein production (see, WO87/05626) but negatively impacts culture viability and reduces cell growth. Up to about 5 mM, preferably less than about 2 mM of butyrate can be added to the medium. However, it is preferred that no butyrate be added to the medium since no advantageous results were seen from adding butyrate. Contrary to others findings no butyrate was necessary in the cell culture environment that includes the high glucose levels of the present invention.

The pH of the medium can be kept constant, e.g., from about 6.5 to about 7.5 during the step, or shifted downward during the culturing. For example, pH can be reduced about 0.05 to about 0.3 pH after, e.g., about 24 to about 96 hours of culturing. Altering the pH of the culture medium in this manner can ordinarily result in less by-product accumulation in non-optimized conditions. In the high glucose-culturing environment described above, however, no significant advantages were determined to be associated with pH shift. In fact, pH shift actually had a negative impact on cell culture productivity by impacting growth negatively. The culture pH can be automatically maintained by the addition of CO₂ (acid) and/or Na₂CO₃ or NaOH (base), as known in the art. The dissolved oxygen concentration can be automatically maintained at 30% of air saturation by direct sparging with air and/or oxygen between 5 and 100%, as is known in the art.

Seeding density range for production culture was about 0.2 to about 0.4% PCV or (1.0×10⁶-3.0×10⁶ Total cells/ml). Higher seeding density (0.4% PCV) was found advantageous with lower final culture temperature (33° C.) whereas lower seeding density (0.2% PCV) was found is advantageous with higher final culture temperature (34° C.). Thus, there was found an interaction between seeding density and final temperature.

Temperature in the growth phase and optional inoculum growth phase is generally maintained within a range of 35° C. and 39° C., and preferably is maintained at 37° C. The initial temperature of the production culture should also preferably be maintained at the same temperature as the prior phase, e.g., 37° C. After a certain period of production, the temperature is preferably shifted down. It is advantageous to use a temperature shift to lower the temperature, since at lower temperatures glucose and lactate metabolism are reduced, and therefore culture viability can be enhanced by delaying the onset of apoptosis.

Where a temperature shift is including in the culturing method, the temperature is preferably shifted down from about 37-39° C. by about 2 to about 8 degrees C., more preferably about 3, 4, 5, or about 6 degrees C. such that the final temperature during the production phase is from about 37° C. to and including about 29° C. The temperature shift can occur at any point after the start of the production phase, and can occur as early as 3 hours after the start of the culturing step and as late as 96 hours, and preferably will occur between 12 and 72 hours, and most preferably will occur between about 24 to about 56 hours after the start of production. Preferred times are about 12, about 24, about 36, about 48, and about 56 hours after the start of the production phase.

To maintain cell viability and productivity, additional quantities of some production medium components, in the form of a concentrated nutrient mixture, termed a “batch feed,” can be fed to the production vessel at specified times during culturing. A batch feed is preferably the source of additional glucose during the production phase. Batch feed should be added at a time after which additions of glucose will have positive effects, and generally at least 12 hours after initiation of the culturing phase. For example, batch feed is preferably added between 12 and 120 hours after the start of culturing. Preferably, batch feed will be added in at least two increments during the culturing phase. These increments are most preferably added between 24 and 72 hours and between 80 and 120 hours, respectively, after initiation of the production phase. Batch feed additions may be effected through addition of concentrated batch feed in the form used in the initial culturing, or modified to remove components. The amount added should range between 1.5 times the original amount used in the culture medium to about 2.5 times this amount. In addition, batch feed may be added which contains no glutamine, or which has a reduced glutamine content.

4. Polypeptide Purification

The polypeptide of interest preferably is recovered from the culture medium as a secreted polypeptide, although it also may be recovered from host cell lysates when directly expressed without a secretory signal. A preferred polypeptide produced by the method of the invention is an antibody, more preferably a monoclonal antibody.

As a first step, the culture medium or lysate is centrifuged to remove particulate cell debris. The polypeptide thereafter is purified from contaminant soluble proteins and polypeptides, with the following procedures being exemplary of suitable purification procedures: by fractionation on immunoaffinity or ion-exchange columns; ethanol precipitation; reverse phase HPLC; chromatography on silica or on a cation-exchange resin such as DEAE; chromatofocusing; SDS-PAGE; ammonium sulfate precipitation; gel filtration using, for example, Sephadex G-75; and protein A Sepharose columns to remove contaminants such as IgG. A protease inhibitor such as phenyl methyl sulfonyl fluoride (PMSF) also may be useful to inhibit proteolytic degradation during purification. One skilled in the art will appreciate that purification methods suitable for the polypeptide of interest may require modification to account for changes in the character of the polypeptide upon expression in recombinant cell culture. In a preferred example, antibodies can be generally purified using chromatographic techniques (e.g., protein A, affinity chromatography with a low pH elution step and ion exchange chromatography to remove process impurities).

### EXAMPLES

The following examples of the practice of the invention are presented by way of illustration and not by way of limitation.

### Materials and Methods

A Chinese hamster ovary (CHO) cell line, derived from dihydrofolate reductase minus (dhfr⁻) DUKX CHO host, was used in all examples (See Urlaub G., Chasin L. A. Isolation of Chinese Hamster Cell Mutants Deficient in Dihydrofolate Reductase Activity, Proc. Natl. Acad. Sci. USA, 77: 41216-4220). In the present examples, CHO cells were genetically engineered to secrete recombinant anti-tissue factor antibody using a dhfr/methoxrexate selection method similar to that used by Kaufman and Sharpe (See Kaufman R. J., Sharpe P. A. Amplification and Expression of Sequences Cotransfected with a Modular Dihydrofolate Reductase Complementary DNA Gene. J. Mol. Biol. 1982, 159, 601-621).

As discussed above, the process of culturing cells involved two or three phases. An initial growth phase where a seed train is produced, an optional inoculum train in which cells are expanded for the inoculum, and a culturing phase in which polypeptide (e.g., antibody) production occurred. Serum-free low-protein (recombinant human insulin) cell culture growth media, based on a mixture of HAM'S F12/DMEM was used in all procedures for these examples. The two basic media formulations (Medium A versus Medium B) have similar compositions as indicated in Table 1 below and were used in the experiments disclosed herein. The levels of some of the components, specifically glutamine and glutamate, were varied during the experiments as described in these Examples.

TABLE 1
Compositions of Media A and B

| | | MEDIUM A | MEDIUM B |
|---|---|---|---|
| Media Components | | mg/L | mg/L |
| 1 | CALCIUM CHLORIDE, | 233.22 | 110.99 |
| | ANHYDROUS | | |
| 2 | CUPRIC SULFATE, | 0.03 | 0.01 |
| | PENTAHYDRATE | | |
| 3 | FERRIC NITRATE, NONAHYDRATE | 0.10 | — |
| 4 | FERROUS SULFATE, | 0.83 | 13.90 |
| | HEPTAHYDRATE | | |
| 5 | POTASSIUM CHLORIDE | 1518.00 | 1492.00 |
| 6 | MAGNESIUM SULFATE, | 97.67 | — |
| | ANHYDROUS | | |
| 7 | MAGNESIUM CHLORIDE, | 286.10 | 142.85 |
| | ANHYDROUS | | |
| 8 | SODIUM PHOSPHATE, | 125.00 | 207.02 |
| | MONOBASIC, MONOHYDRATE | | |
| 9 | SODIUM PHOSPHATE, DIBASIC, | 142.04 | — |
| | ANHYDROUS | | |
| 10 | ZINC SULFATE, HEPTAHYDRATE | 0.86 | 2.16 |
| 11 | HYPOXANTHINE (Na) | 33.39 | — |
| 12 | LINOLEIC ACID | 0.59 | 0.70 |
| 13 | LIPOIC ACID | 1.47 | 0.31 |
| 14 | PUTRESCINE, DIHYDROCHLORIDE | 1.13 | 1.13 |
| 15 | SODIUM PYRUVATE | 770.00 | 388.68 |
| 16 | THYMIDINE | 5.11 | — |
| 17 | ALANINE | 62.30 | 13.36 |
| 18 | ARGININE, | 1561.00 | 316.05 |
| | MONOHYDROCHLORIDE | | |
| 19 | ASPARAGINE, MONOHYDRATE | 641.97 | 1125.98 |
| 20 | ASPARTIC ACID | 511.60 | 665.50 |
| 21 | CYSTEINE, | 245.84 | 351.20 |
| | MONOHYDROCHLORIDE, | | |
| | MONOHYDRATE | | |
| 22 | CYSTINE, DIHYDROCHLORIDE | 62.57 | — |
| 23 | GLUTAMIC ACID | 102.90 | 147.10 |
| 24 | GLUTAMINE | 1606.00 | 2191.50 |
| 25 | GLYCINE | 370.90 | — |
| 26 | HISTIDINE, | 188.72 | 314.40 |
| | MONOHYDROCHLORIDE, | | |
| | MONOHYDRATE | | |
| 27 | ISOLEUCINE | 397.78 | 393.60 |
| 27 | LEUCINE | 589.90 | 656.00 |
| 29 | LYSINE, MONOHYDROCHLORIDE | 2207.80 | 456.63 |
| 30 | METHIONINE | 184.16 | 223.80 |
| 31 | PHENYLALANINE | 100.72 | 330.40 |
| 32 | PROLINE | 241.50 | 230.20 |
| 33 | SERINE | 634.80 | 262.75 |
| 34 | THREONINE | 839.10 | 238.20 |
| 35 | TRYPTOPHAN | 90.68 | 153.15 |
| 36 | TYROSINE, DISODIUM SALT, | 273.15 | 394.80 |
| | DIHYDRATE | | |
| 37 | VALINE | 527.50 | 351.30 |
| 38 | BIOTIN | 0.051 | 0.02 |
| 39 | D-CALCIUM PANTOTHENATE | 7.36 | 7.15 |
| 40 | CHOLINE CHLORIDE | 101.72 | 209.40 |
| 41 | CYANOCOBALAMIN | 9.52 | 13.55 |
| 42 | FOLIC ACID | 19.84 | 6.62 |
| 43 | i-INOSITOL | 133.20 | 90.10 |
| 44 | NIACINAMIDE | 4.26 | 6.11 |
| 45 | PYRIDOXAL, | 4.00 | — |
| | MONOHYDROCHLORIDE | | |
| 46 | PYRIDOXINE, | 0.43 | 0.21 |
| | MONOHYDROCHLORIDE | | |
| 47 | RIBOFLAVIN | 0.67 | 0.19 |
| 48 | THIAMINE, | 6.38 | 3.37 |
| | MONOHYDROCHLORIDE | | |

| | | |
|---|---|---|
| | OTHER TRACE ELEMENTS | |
| | (included only in GEM powder-otherwise from trace | |
| | element solution): | |
| 49 | AMMONIUM PARAMOLYBDATE, TETRAHYDRATE | 0.004 |
| 50 | AMMONIUM VANADIUM OXIDE | 0.002 |
| 51 | MANGANESE SULFATE, MONOHYDRATE | 0.0002 |
| 52 | NICKEL CHLORIDE, HEXAHYDRATE | 0.0004 |
| 53 | SELENIOUS ACID | 0.012 |
| 54 | SODIUM METASILICATE, NONAHYDRATE | 0.43 |
| 55 | STANNOUS CHLORIDE, DIHYDRATE | 0.0003 |

During the growth phase, the cells were continuously subcultivated in log phase under selective pressure in a seed train. The cells from the seed train were expanded in bioreactors using nonselective medium to provide inoculum for production cultures. 3L liter glass stirred bioreactors (Applikon, Foster City, Calif.) were used for both inoculum and production cultures at 1.5-2.0L working volume. Temperature, pH, agitation and dissolved oxygen were controlled using digital control units (B. Braun Biotech International, Allentown, Pa.). Temperature in seed and inoculum cultures was maintained at 37° C. in the principle experiment. The initial temperature of the production culture was also maintained at 37° C.

After a specified period following inoculation, the temperature was shifted down to the levels specified in the experimental design and the temperature was maintained at that set point for the duration of the culture.

The culture pH was automatically maintained by the addition of CO₂ (acid) and/or Na₂CO₃ or NaOH (base), as known in the art. The dissolved oxygen concentration was automatically maintained at 30% of air saturation by direct sparging with air and/or oxygen as known. To maintain cell viability and productivity, additional quantities of some production medium components, in the form of a concentrated nutrient mixture (batch feed), were fed to the production vessel at specified times. Batch feed had some initial media components in concentrated form (4 fold concentrated). After addition of the batch feed to the production phase culture medium, the initial media components were increased by 30-40%, including a 30% increase in initial peptone concentration, an increase of greater than 100% glucose concentration, and an increase of 100% of trace elements.

Daily samples were taken to monitor cell growth, viability and extracellular product titer. Cell growth was determined by cell counts and packed cell volume (PCV). PCV was measured using graduated centrifuge tubes (Kimble Science Products, Fullerton, Calif.) after spinning cell suspension for 10 minutes at 830 g. PCV was expressed as percentage of the total culture volume. Cell viability was determined by a standard trypan-blue dye-exclusion method. The pH of the culture, Na⁺, K⁺, lactate and glucose concentrations in supernatant were measured using Nova-Bioprofile® (Nova Biomedical, Waltham, Mass.) according to the manufacturer's instructions. Product titer was quantified with HPLC by using a Protein A column (POROS® 20A, Perkin Elmer) to quantitate Fc-containing anti-tissue factor antibody in cell culture supernatant. Product quality of selected samples was determined by measuring the number of terminal galactose residues per glycan using MALDI-TOF mass spectrometry (see, for example, Papac, D. I. et al., Analysis of acidic oligosaccharides and glycopeptides by matrix-assisted laser-desorption ionization time-of-flight mass spectrometry. Anal. Chem. 68:3215-3223 (1996)). Alternately, CE-LIF (capillary electrophoresis and laser-induced flouorescence) may be used to analyze glycan terminal galactosylation.

### Experimental Design

Eleven cell culture parameters were examined in this study: final temperature after a temperature shift, temperature shift timing, butyrate concentration, media type (A or B), pH shift, batch feed (one or more batch feedings), glutamine concentration, glutamate concentration, total amount of glucose added to the medium throughout the experiment, glucose regimen (e.g., the number of increments, amount, and timing of glucose addition) and the seeding density. Full factorial designs were generated using JMP® software (SAS Institute, 1996, supra). Preferred levels and ranges for these factors are shown in Table 1.

In the first set of experiments, four factors (final temperature, temperature shift timing, butyrate and media) were studied at two levels. A repeated full factorial design required 2×2ⁿ=32 runs for n=4 factors. The experiments were performed in blocks. In the design of eight blocks of size four, blocking effect (performing experiments sequentially rather than all at once) was confounded with the two way interaction of temperature shift timing with butyrate.

The second set of experiments was performed after analyzing the results of the first set of experiments. Temperature, temperature shift timing, butyrate and media were set at 34° C., 24 hrs, 0 mM butyrate and media (A) respectively while investigating the effects of pH shift, glutamine levels and batch feed additions. Two levels were identified for pH shift and batch feed. The pH set point was lowered from 7.15 to 7.0 on day 3 of fermentation for the pH shifted cases only. All cell cultures in this set of experiments received additional quantities of the same production medium components (except glutamine) in the form of a concentrated mixture (batch feed) on day 2. Glutamine was omitted from the batch feed to investigate its influence as a separate factor. The cultures with two batch feeds received the second feed with the same composition on day 4 or 5. All cases had an initial glutamine level of 5 mM. Additional 5 mM glutamine was added from a concentrated glutamine stock on day 2 for 10 mM glutamine cultures. Cultures containing 15 mM glutamine received glutamine additions on both days 2 and 5. The mixed level fill factorial design for this set of experiments required 2ⁿ×3^{k}=12 runs where n is the number of 2 level factors and k is 3 level factors.

In the third set of experiments, the impact of additional glucose (compared to the previous set of experiments), multiple additions of batch feed (two batch feed additions in these experiments), and glutamine as well as their affects on osmolality and final product titer was investigated. A total of twelve runs were performed for this set of experiments.

In the fourth set of experiments, effects of seeding density, replacement of glutamine with glutamate, glucose addition regimen, and additional ranges of temperature were examined. As described, supra, the results of the third set of experiments indicated that glucose in the initial media, in first batch feed, and further provided by addition on day 4, had a favorable affect. Thus, in the fourth set of experiments, the effect of glucose regimen was examined: in the “no additional glucose” regimen, experimental cell cultures received no additional glucose other than that provided in the initial media (‘no additional’); in the “high glucose” regimen, cultures received 10 g/L additional glucose on day 4 (‘high’); and in the “multiple glucose” regimen, cultures received 6 g/L on day 4 plus 4 g/L on day 7 (‘multiple’). A total of 24 runs were performed for this set of experiments.

TABLE 2
Range and levels of cell culture variables.

| Ex- | | | | |
|---|---|---|---|---|
| peri- | | | | |
| ment | Variable | Lower Level | Intermediate | Higher Level |
| 1^{st} | Temperature | 31° C. | — | 34° C. |
| set | Temp shift | 24 hrs | — | 56 hrs |
| | time | | | |
| | Butyrate | 0 mM | — | 1.5 mM |
| | Media | A | — | B |
| 2^{nd} | pH shift | No shift (set at | — | Shift to 7.0 on |
| set | | 7.15) | | day 3 |
| | Batch feed | Once (day 2) | — | Twice (day 2 and |
| | | | | 5) |
| | Glutamine | 5 mM | 10 mM | 15 mM |
| 3^{rd} | Glucose | No addition | — | 10 g/L on day 4 |
| set | Batch feed^{b} | Once (day 2) | — | Twice (day 2 and |
| | | | | 4) |
| | Glutamine | 5 mM | 10 mM | 15 mM |
| 4^{th} | L-glutamine/L- | 5 mM L- | — | 5 mM L- |
| set | glutamate | glutamate | | glutamine |
| | Seeding density | 0.2% PCV^{a} | — | 0.4% PCV |
| | Temperature | 33 | | 35 |
| | Glucose | no addition | multiple | High |
| | regimen | | | |

^{a}PCV refers to packed cell volume.

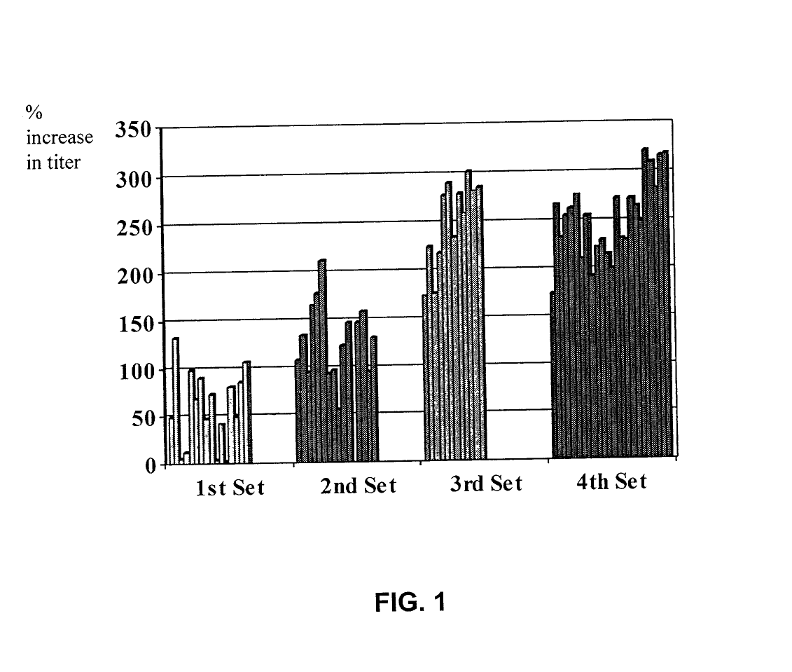

Experiments were conducted according to Table 1. The experimental results are reported for protein or antibody product titer, cell growth in terms of packed cell volume (PCV), culture viability, and by-product accumulation (lactate and ammonium). Calculated results (integrated viable PCV, specific productivity based on viable PCV) were analyzed using JMP® software. The models included select two-way and three-way interactions. FIG. 1 summarizes the general trend in improved product titer as a result of cell culture optimization from the first set of experiments to the fourth set of experiments. Product titer (such as antibody titer) was measured by an HPLC assay. Titer increase was calculated by:

% increase in titer=((Improved titer-initial titer)/initial titer)×100

### Example 1

### Results from the First Set of Experiments

The analysis of the first set of experiments identified temperature and media as having significant effects. Interactions were found between final temperature and temperature shift timing and also final temperature and media. Tables 2 and 3 show the scaled parameter estimates (estimated value for each factor in the model), standard error (SE), T ratio (ratio of parameter estimate to standard error) and the P-values for titer and % final culture viability responses respectively. Small P values indicate that the mean response varies when the associated variable is altered. A P value below 0.05, by convention, is considered statistically significant. An F value is calculated by dividing the mean square model error by the mean square error variance. F value provides information on how well the factors describe the statistical variation in the data from its mean. The higher the F value from unity, the greater is the certainty of the factors explaining the variation in the data about its mean.

In the present invention, the F values for both responses between final temperature and temperature shift timing and also final temperature and media (Tables 3 and 4) indicate that the temperature variation at specified times as described herein is significant. Table 3 shows a very small P-value for temperature shift, indicating that it has a very significant main effect on product antibody titer. In particular, a temperature shift from the initial temperature of 37° C. down to a range between 31 to 34° C. was significant when said shift occurred between 12 and 36, and preferably at about 24 hours after culturing. The positive parameter estimate shows that the titer increases in proportion to the higher end of the temperature range for the post-shift temperature. The interaction between time and temperature (denoted as “time*temperature”) was highly significant. Large negative temperature*time interaction suggests that using the low temperature shifting time (24 hours) with the high end of the post-shift temperature range (i.e., 33 to 34 C.) provides the best titer. The specific time*temperature relationship was also dependent on the seeding density. In particular, the highest titers were observed in a 0.2% seeding density where the shift occurred at 24 hours and moved from 37° C. to 34° C., while in a 0.4% seeding density a shift at 24 hours from 37° C. to 33° C. produced the highest titers. The block effect (performing experiments in blocks) was insignificant.

The media used in such settings did not appear to affect titer but did affect viability of the cell culture medium (Table 4). In particular, media that had higher glutamine concentrations, including as a consequence of batch feed additions containing glutamine) had lower viability. Higher ammonia levels accumulated in cultures with higher glutamine concentrations. This is consistent with the negative impact of ammonium on growth, viability, and product quality reported by others (see, e.g., McQueen, A., Bailey, J. E., Mathematical Modeling of the Effects of Ammonia ion on the Intracellular pH of hybridoma Cells., 1990, Biotechnology and Bioengineering, 35:897-906;. Wu, P., Ray, N. G., and Shuler, M. L., A Computer model for intracellular pH Regulation in Chinese Hamster Ovary Cells, 1993, Biotechnol. Prog., 9:374-384; and Wu, P., Ray, N. G., and Shuler, M. L., A Single-Cell Model for CHO Cells. 1992, Ann. N. Y. Acad. Sci. (Biochem. Eng. VII), 152-187). Thus, the lower viability of media was due to the additional glutamine in such media.

TABLE 3
Analysis of data from first experiment. Response-Titer

| | Parameter | | | |
|---|---|---|---|---|
| Model Variable | Estimate | SE | T Ratio | P-value |
| Intercept | 316.25 | 8.13 | 38.91 | 0.000 |
| Temperature | 60.68 | 8.13 | 7.47 | 0.000** |
| Media | 4.375 | 8.13 | 0.54 | 0.596 |
| Temp. shift time | 16.25 | 8.13 | 2.0 | 0.0587 |
| Butyrate | −15.81 | 8.13 | −1.95 | 0.0652 |
| Temp * Media | −3.31 | 8.13 | −0.41 | 0.6877 |
| Temp * time | −28.82 | 8.13 | −3.55 | 0.0019** |
| Temp * Butyrate | −10.625 | 8.13 | −1.31 | 0.2052 |
| Media * time | 9.25 | 8.13 | 1.14 | 0.2679 |
| Media * Butyrate | 13.5625 | 8.13 | 1.67 | 0.11 |
| Block effect | −0.1875 | 8.13 | −0.02 | 0.9818 |

*Block effect is confounded with the interaction between time and butyrate.
**Statistically significant, P < 0.05.
Residual standard error: 51.993 on degrees 21 of freedom
F-stat: 8.24 P-value: 0.001

TABLE 4
Statistical analysis of data from first experiment.
Response = % final viability

| | Parameter | | | |
|---|---|---|---|---|
| Model Variable | Estimate | SE | T Ratio | Prob > T |
| Intercept | 80.300 | 2.013 | 39.91 | 0.000 |
| Temperature | −6.475 | 2.013 | −3.22 | 0.004** |
| Media | −9.68 | 2.013 | −4.81 | 0.000** |
| Temp. shift | −2.75 | 2.013 | −1.37 | 0.186 |
| time | | | | |
| Butyrate | −0.713 | 2.013 | −0.35 | 0.727 |
| Temp * Media | −4.82 | 2.013 | −2.39 | 0.026** |
| Temp * time | 1.337 | 2.013 | 0.66 | 0.513 |
| Temp * Butyrate | −1.175 | 2.013 | −0.58 | 0.566 |
| Media * time | −2.993 | 2.013 | −1.491 | 0.151 |
| Media * Butyrate | −0.443 | 2.013 | −0.22 | 0.828 |
| Time * Butyrate | 1.537 | 2.013 | 0.76 | 0.453 |

Residual standard error: 11.405 on degrees 21 of freedom
F-stat: 4.48 P-value: 0.0018
**Statistically significant, P < 0.05.

### Example 2

### Results from the Second Set of Experiments

A second set of experiments investigated the impact of varying glutamine levels in addition to pH and batch feed. Glutamine was removed from the batch feed composition to separate the effects of batch feed and glutamine. The best temperature (34° C.), temperature shift timing (24 hrs), butyrate concentration (0 mM) and Media (A) combination deduced from the first set of experiments was used as a baseline for the second set of screening experiments. The results in Table 5 show that both batch feed and pH had the most significant main affects on titer.

TABLE 5
Statistical Analysis of data from second experiment, Response: Titer

| Coefficient | Est | SE | T(b = 0) | Prob > \|T\| |
|---|---|---|---|---|
| intercept | 471.500 | 21.568 | 21.861 | 0.000 |
| Glutamine | −38.125 | 21.568 | −1.768 | 0.120 |
| Glutamine\hat{ }2 | −63.875 | 30.502 | −2.094 | 0.075 |
| pH | −59.438 | 15.251 | −3.897 | 0.006 ** |
| Batch feed | 32.813 | 15.251 | 2.152 | 0.068 ** |
| Glutamine * pH | −53.875 | 21.568 | −2.498 | 0.041 ** |
| Glutamine * Batch feed | −11.875 | 21.568 | −0.551 | 0.599 |
| pH * Batch feed | −23.938 | 15.251 | −1.570 | 0.161 |
| Glutamine * pH * Batch feed | −41.125 | 21.568 | −1.907 | 0.098 |

Residual standard error: 61.003 on degrees 7 of freedom
F test of Null Model: F-stat: 4.996 P-value: 0.024

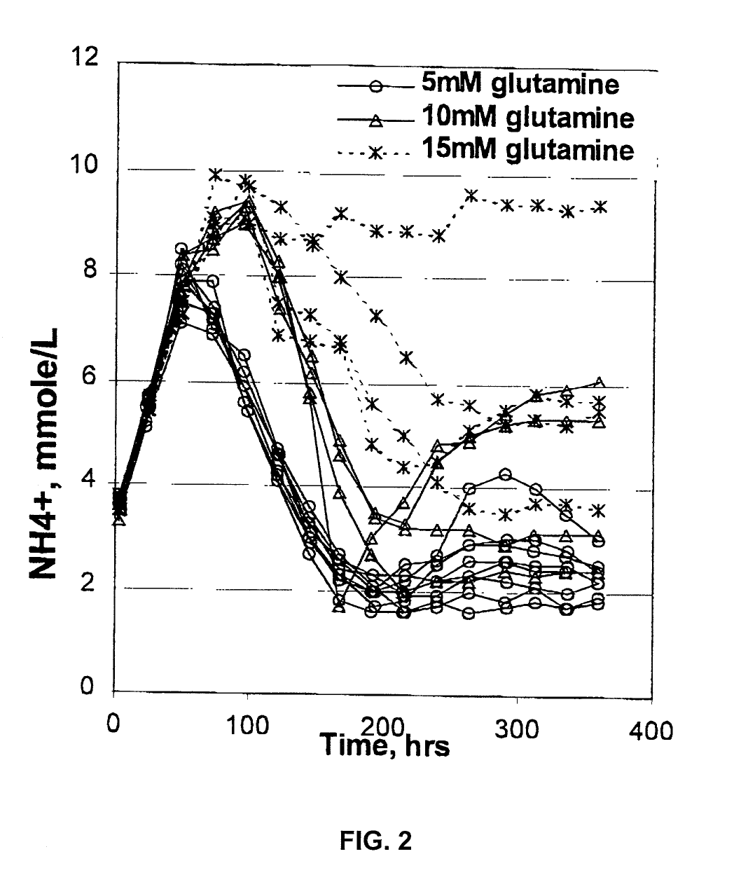

The factor of pH shift had a negative impact while batch feed had a positive impact on titer. Therefore, it is preferred to not reduce pH during the culturing or production phases. Evidence for the main effect of glutamine on titer was not strong; however glutamine appeared to interact with pH. FIG. 2 shows that highest maximum ammonium level correlates with highest glutamine cases (15 mM). Ammonium levels decrease in all cases as the culture progresses. There are reports in the literature that high ammonium levels lower intracellular pH. For example, one model for CHO cells predicted a severe decrease in pH in the presence of ammonium, both endogenous and exogenous, even at the moderate level of a few millimolar ammonium (see Wu et al., 1993 and 1992, supra).

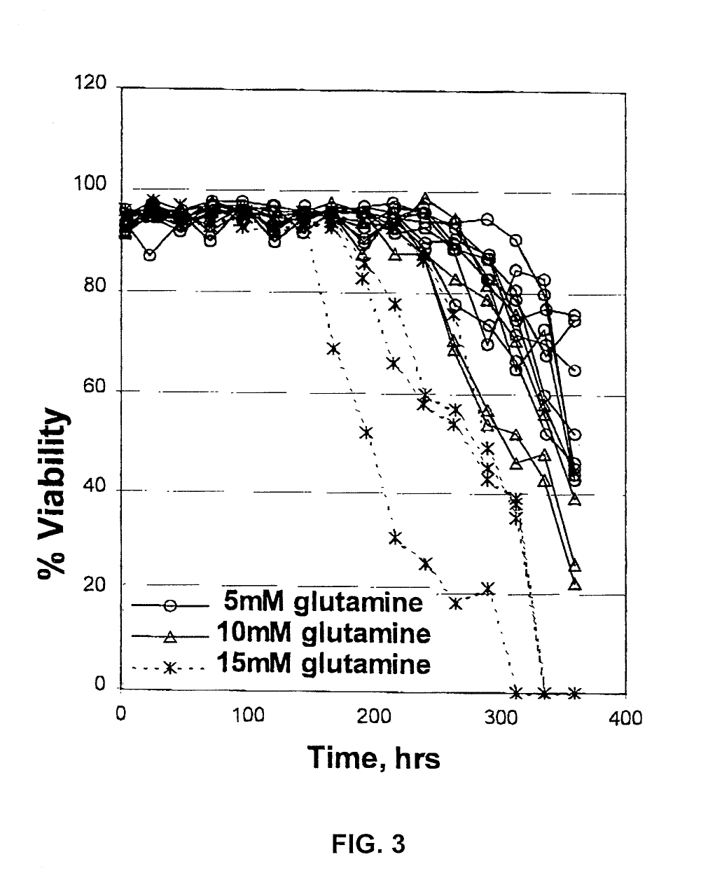

FIG. 3 shows a negative impact on viability with increasing glutamine levels. A significant decrease in CHO culture viability in relation to the higher ammonium levels was reported in literature by other researchers (see Yang, M., and Butler, M. Effect of Ammonia on the Glycosylation of Human recombinant Erythropoietin in Culture, Biotechnol. Prog., 2000, 16(5):751-759). The present results are consistent with this.

Higher osmolality was measured in the cultures with high glutamine and second batch feed addition. The glucose component of the batch feed was one of the contributors to higher osmolality. Glutamine also impacted osmolality due to higher lactate production with higher glutamine levels and higher base addition to these cases to control the pH. Cultures with higher osmolality had higher volumetric titers. See FIG. 1. This finding is consistent with reports in literature showing an increase in specific productivity with increasing osmolality (See, for example, Chen, M. and Forman, L. W., Polypeptide Production in Animal Cell Culture. 1999, and U.S. Pat. No. 5,856,179).

### Example 3

### Results of the Third Set of Experiments

As shown herein, osmolality is one of the significant variables in cell culture processes. It can be impacted by various parameters. For example, media components, especially glutamine and glucose, impact osmolality by their concentration levels or by affecting the accumulation of by-products lactate and ammonia.

The effects of glucose batch feeding and glutamine concentration on osmolality and product titer was investigated in more detail in a third set of experiments. All cell cultures were maintained at a glucose concentration of at least about 3 g/L to promote cell viability. In the test cultures, glucose additions on day 4 or 5 were primarily used to increase osmolality. In these experiments, glucose was omitted from the second batch feed only and glutamine was omitted from both batch feeds. All cultures had an initial 5 mM glutamine level and later additions of glutamine and glucose were made using concentrated glutamine and glucose solutions.

Table 6 shows the results of data analysis for titer response for this third set of experiments. A very small P-value calculated for glucose indicates that it is a significant factor for the titer response. A positive parameter estimate shows that addition of glucose impacts the response in a positive way. Increasing the number of batch feeds also had a positive impact on titer. In these experiments, batch feed was added once (day 2) or twice (day 2 and day 4 or 5), and it was observed that two batch feed additions had statistically significant beneficial effects. In these expermiments, glutamine did not have a significant impact on titer.

TABLE 6
Variable_analyzed = Titer (day 15) for the third set of experiments

| Coefficient | Estimate | SE | T Ratio | Prob > T |
|---|---|---|---|---|
| intercept | 730.75 | 25.768 | 23.86 | <.0001 |
| L-glutamine | 20.75 | 18.221 | 1.14 | 0.2923 |
| L-glut quad | −47.5 | 31.56 | −1.51 | 0.1760 |
| batch feed | 46.583 | 14.877 | 3.13 | 0.0166** |
| glucose | 51.25 | 14.88 | 3.44 | 0.0108** |

Residual standard error: 51.538 on degrees 7 of freedom
F test of Null Model: F-stat: 6.308 P-value: 0.018

The results of this third set of experiments also showed a very significant negative linear correlation between cell culture viability and glutamine levels. Batch feed seemed to have a significant positive impact on viability. Glucose and batch feed had positive impact on maximum specific productivity (Table 7). Other factors contribute to the culture's osmolality besides the factors examined; namely, pH control and dissolved carbon dioxide levels. Batch feed does not impact osmolality as much when its glucose component was removed. Osmolality increases upon glucose addition, however osmolality decreases as glucose is metabolized

TABLE 7
Variable_analyzed = Specific Productivity (Titer/cell/day)

| Coefficient | Est | SE | T(b = 0) | Prob > \|T\| |
|---|---|---|---|---|
| Intercept | 0.140 | 0.003 | 41.173 | 0.000 |
| L-glut lin | 0.000 | 0.004 | 0.090 | 0.931 |
| L-glut quad | −0.005 | 0.007 | −0.744 | 0.481 |
| Batch feed | 0.008 | 0.003 | 2.350 | 0.051** |
| Glucose | 0.011 | 0.003 | 3.231 | 0.014** |

Residual standard error: 0.012 on degrees 7 of freedom
F test of Null Model: F-stat: 4.131 P-value: 0.050
**Statistically significant, P < 0.05.

### Example 4

### Results of the Fourth Set of Experiments

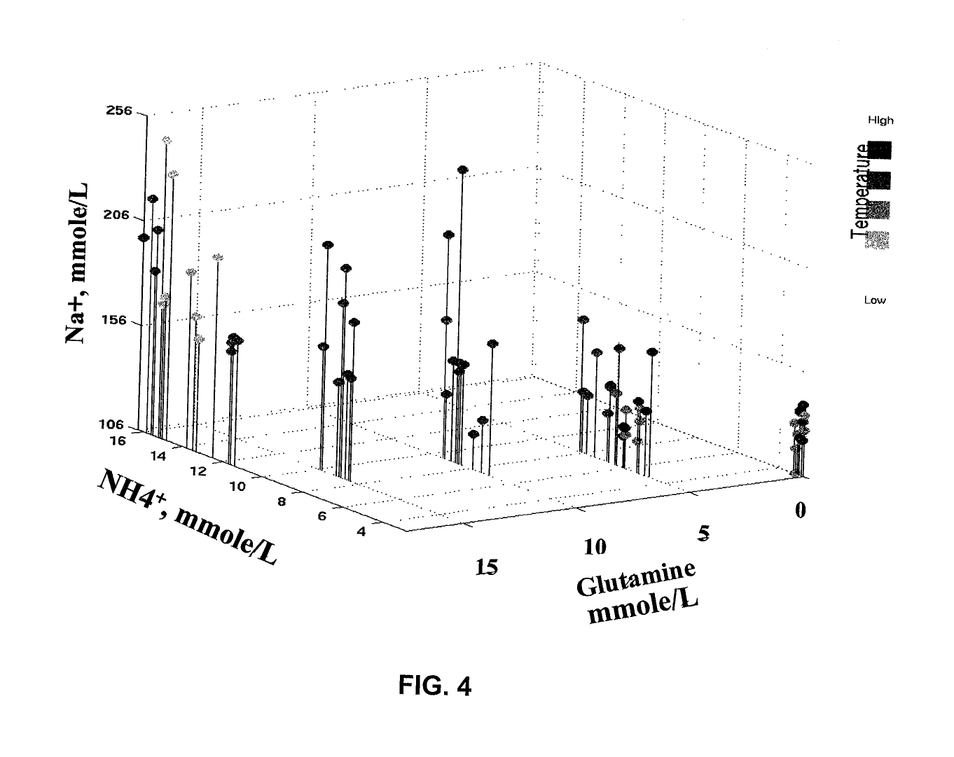

A fourth set of experiments investigated by-product accumulation by examining the use glutamate as an alternative to glutamine in the cell culture medium. Seed density, final temperature (using a different range of temperatures compared to the first experiment), and the glucose regimens were other factors investigated. Significant individual effects for seed density, glutamine versus glutamate, temperature and glucose regimens were determined for titer response. Seed density was also noted to interact with temperature. Lower temperature with higher seeding density is preferred for higher product titers. If the seeding density is low, however, temperature has little effect. FIG. 4 summarizes the accumlation of by-products ammonium ion and lactate for all the experiments in this fourth set of experiments. Maximum ammonium levels are shown on the y axis. On the z-axis (vertical bars), Na+ concentration represents the concentration of added Na₂CO₃ required to neutralize lactate as a means for controlling pH in the medium, thus allowing the Na+ concentration to reflect the amount of lactate produced during the production phase. Glutamine concentrations are indicated on the x-axis. The final glutamine concentration in the production phase varies, right to left on the x-axis, from 0 mM glutamine, to 5 mM glutamine, to 10 mM glutamine (5 mM initial concentration, with glutamine added as batch feed to 10 mM), to 15 mM glutamine (10 mM initial concentration, with glutamine added as batch feed to 15 mM). Glutamate was present in the culture medium at a concentration of 5 mM in the experiment with 0 mM glutamine. Temperature is indicated in the shaded circles atop the vertical bars as shades of grey, where dark grey representing 35° C. as “high” temperature, while the lightest grey represents “low temperature” of 31° C. Intermediate shades of darker and lighter grey represent 34° C. and 33° C., respectively. Each vertical bar represents the results of an individual experiment in this fourth set of experiments. Thus, FIG. 4 shows that 5 mM glutamate (at 0 mM glutamine) leads to the lowest level of by-product accumulation (shortest vertical bars at low NH₄⁺ concentration) regardless of the culture temperature. Other researchers have reported generation of minimal quantities of lactate and ammonium upon substitution of glutamine with glutamate (See, Altamirano, C., Paredes, C., Cairo, J. J., and Godia, F., Improvement of CHO Cell Culture Medium Formulation: Simultaneous Substitution of Glucose and Glutamine. Biotechnol. Prog. 2000, 16:69-75).

### Example 5

### Product Quality

Product quality was evaluated for selected antibodies produced by the method of the invention by analyzing the percentage of antibody glycans having zero, one, or two terminal galactose residues. Antibody glycan distribution, and particularly terminal galactosylation, has profound affects on complement recognition, immune modulation by ADCC (antigen-dependent cytotoxic cellular) response, aggregation of the antibody. Preferably, the percentage of glycans having zero galactose residues (G0) is less than about 80%, more preferably less than about 70%, even more preferably less than about 60%, and most preferably about 55% or less.

By-product accumulation adversely affects product antibody quality. One by-product, ammonium ion, had an adverse affect on terminal galactosylation and, as a result, had an adverse affect on antibody quality. Several cell cultures were shown to have varying levels of ammonium accumulation at the end of the production phase. Table 8 shows quality data, as glycan distribution, as a function of ammonium ion accumulation in the production phase culture medium for antibodies isolated from these cultures. The ammonia levels correlated with increasing glutamine concentration and the total amount of glucose added to the production phase culture medium. Each of the ammonium ion levels shown in Table 8 was the maximum level measured throughout the cell culture process. The data indicate a negative impact of increasing ammonia levels on glycan distribution. As the maximum ammonium concentration increased from 4 to 13 mM, a concurrent increase (36%) in the percentage of G0 glycans was observed, thereby indicating a decrease in terminal galactosylation. Other researchers also showed that ammonium in the culture medium affects glycolysation (see Chen, 1999, supra). They reported a significant increase in the heterogeneity of the glycoforms and a reduction of the sialylation at 20 mM NH₄Cl. A 40% decrease in terminal galactosylation was observed as ammonium increased from 1 mM to 15 mM post growth phase with CHO cells, as reported by another group (See Gawlitzek, M., Ryll, T., Lofgren, J., Sliwkowski, M. B., Ammonium Alters N-Glycan Structures of Recombinant TNFR-IgG: Degradative Versus Biosynthetic Mechanisms, Biotechnology and Bioengineering, 2000, 68:637-646).

TABLE 8
Impact of ammonium as by-product on antibody quality.

| Glutamine | | 5 mM | 7.5 mM | 7.5 mM | 10 mM | 15 mM |
|---|---|---|---|---|---|---|
| concen- | | | | | | |
| tration | | | | | | |
| NH₄⁺ | | 4 mM | 6.8 mM | 6.8 mM | 9 mM | 13 mM |
| concen- | | | | | | |
| tration | | | | | | |
| Glycan | G0^{b} | 55% | 64% | 70% | 89% | 91% |
| Distribution^{a} | G1^{c} | 38% | 32% | 27% | 10.6% | 8.6% |
| | G2^{d} | 7% | 4% | 3% | 0.4% | 0.4% |

^{a}Glycan Distribution refers to the percentage of glycans having 0, 1, or 2 terminal galactose residues as measured using MALDI-TOF MS.
^{b}G0 = Zero terminal galactose residues per glycan residue.
^{c}G1 = One terminal galactose residues per glycan residue.
^{d}G2 = Two terminal galactose eresidues per glycan residue.

The present invention allows for reduced levels of glutamine, e.g, less than 5 mM, to be used in a cell culturing medium, thereby reducing lactate and ammonium ion accumulation. Surprisingly, the present invention allows an increased level of glucose without causing an increase in by-product accumulation. The higher glucose levels result in increased osmolality and enhanced polypeptide (e.g. antibody) productivity and antibody quality.

While this invention has been described in connection with specific embodiments thereof, it will be understood that it is capable of further modifications. This application is intended to cover any variations, uses, or adaptations of the inventions following, in general, the principles of the invention and including such departures from the present disclosure as come within known or customary practice within the art to which the invention pertains and as may be applied to the essential features hereinbefore set forth as follows in the scope of the appended claims.

We claim:

1. A method of producing a polypeptide in a cell culture comprising:
(a) growing in a cell culture medium animal cells comprising an isolated nucleic acid encoding a desired polypeptide, and
(b) culturing in a production phase the animal cells in a cell culture medium such that the cells express the polypeptide, wherein glucose is added to the medium at the beginning of said culturing or during said culturing to create a glucose concentration in the medium of greater than about 10 g/L.

2. The method of claim 1, further comprising between said growing and said culturing, expanding the animal cells to provide an inoculum and adding the inoculum to the cell culture medium at the beginning of the culturing.

3. The method of claim 1, wherein at some point of said culturing, the glucose concentration in the medium is at least about 12 g/L up to and including about 40 g/L.

4. The method of claim 1, wherein at some point of said culturing, the glucose concentration in the medium is equal to or greater than a concentration selected from the group consisting of about 12, about 13, about 14, about 15, about 16, about 17, about 18, about 19, or about 20 g/L.

5. The method of claim 1, wherein the total amount of glucose added at the beginning and during said culturing is from about 10 g/L to and including about 40 g/L.

6. The method of claim 1, wherein the total amount of glucose added at the beginning and during said culturing is from about 15 to and including about 30 g/L.

7. The method of claim 5, wherein glucose is added in two or more increments during said culturing, either alone or as part of batch feed.

8. The method of claim 1, wherein at the start of said culturing the cell culture medium has an osmolality of about 280 to about 380 mOsm, and at some point of said culturing the culture medium has an osmolality of about 400 to about 600 mOsm.

9. The method of claim 1, wherein at the start of said culturing, the cell culture medium has an osmolality of about 300 to and including about 350 mOsm and at some point of said culturing has an osmolality of about 420 to and including about 500 mOsm

10. The method of claim 1, wherein said culturing begins at least 3 hours after the beginning of said growing.

11. The method of claim 1, wherein said culturing begins at least 24 hours after the beginning of said growing.

12. The method of claim 1, wherein said culturing begins at a time from about 48 to and including about 192 hours after the beginning of said growing.

13. The method of claim 1, wherein glucose concentration is maintained in said culturing to be greater than at least about 2 g/L until glucose is added to create a glucose concentration in the medium greater than about 10 g/L.

14. The method of claim 1, wherein the concentration of glutamine in the medium during said culturing is less than about 5 mM.

15. The method of claim 1, wherein no glutamine is added to the medium during said culturing.

16. The method of claim 1, wherein glutamate is added to the medium during said culturing such that the concentration of glutamate is from about 1 to and including about 10 mM glutamate.

17. The method of claim 1, wherein during said culturing the ratio of glutamate concentration to glutamine concentration in the cell culture medium is at least 2:0.5.

18. The method of claim 1, wherein a batch feed comprising glucose is added to the cell culture medium during said culturing in one or more increments.

19. The method of claim 1, wherein a batch feed comprising glucose is added to the cell culture medium at least twice during said culturing.

20. The method of claim 1, wherein a batch feed comprising glucose is added to the cell culture medium between about 3 hours and about 120 hours after the start of said culturing

21. The method of claim 19, wherein a batch feed comprising glucose is added to the cell culture from about 24 to and including about 72 hours, and again from about 80 to and including about 120 hours, after the start of said culturing.

22. The method of claim 1, wherein during said culturing, the temperature of the medium is reduced by at least 2 degrees C. relative to the temperature at the beginning of said culturing.

23. The method of claim 22, wherein the temperature is reduced from about 35 to 39 degrees C. at the beginning of said culturing to about 31 to 35 degrees C. for the remainder of said culturing.

24. The method of claim 22, wherein the temperature is reduced at a time at about 12 hours to about 72 hours after the beginning of said culturing.

25. The method of claim 22, wherein the temperature is reduced at least about 2, at least about 3, at least about 4, or at least about 5 degrees C., at a time selected from the group consisting of at least about 12 hours, at least about 24 hours, at least about 36 hours, at least about 48 hours, and at least about 56 hours, after the beginning of said culturing.

26. The method of claim 1, wherein at the beginning of said culturing an inoculum having a seeding density of about 0.2% PCV is added to the culture medium, and wherein the temperature of the culture medium is reduced at about 24 hours after beginning said culturing, wherein the temperature is reduced from about 37° C. to about 34° C.

27. The method of claim 1, wherein at the beginning of said culturing an inoculum having a seeding density of about 0.4% PCV is added to the culture medium, and wherein the temperature of the culture medium is reduced at about 24 hours after the beginning of said culturing, wherein the temperature is reduced from about 37° C. to about 33° C.

28. The method of claim 1, wherein at the beginning of said culturing the cells in the culture medium have a seeding density from about 0.1 to about 0.5% PCV.

29. The method of claim 1, wherein the cell culture medium is free of serum in the growing and culturing.

30. The method of claim 1, wherein the cell culture medium is free of animal-derived proteins in both the growing and culturing.

31. The method of claim 1, wherein the cells are mammalian cells.

32. The method of claim 1, wherein the cells are Chinese Hamster Ovary cells.

33. The method of claim 1, wherein the polypeptide is a monoclonal antibody.

34. The method of claim 33, wherein the monoclonal antibody is selected from the group consisting of anti-HER2, antibody 2C4, anti-VEGF, antibody C2B8, anti-CD11a, anti-tissue factor, IgG4b, anti-CD40, anti-CD20, anti-IgE, antibody E25, and antibody E26.

35. The method of claim 1, wherein no butyrate is added during said culturing, and the culture medium is maintained at a pH between about 6.5 and about 7.5 during said culturing.

36. The method of claim 1, wherein during said growing, the culture medium comprises about 1 to about 12 g/L of glucose, and wherein during or at the beginning of said culturing, at total of greater than 10 g/L of glucose is added to the culture medium.

37. A monoclonal antibody produced by the method of claim 1.

38. The monoclonal antibody of claim 37, wherein the antibody comprises at least one glycan moiety and the percentage of glycans having zero terminal galactose residues in a population of said antibodies is less than about 80%.

39. The monoclonal antibody of claim 37, wherein the antibody comprises at least one glycan moiety and the percentage of glycans having zero terminal galactose residues in a population of said antibodies is less than about 70%.

40. The monoclonal antibody of claim 37, wherein the antibody comprises at least one glycan moiety and the percentage of glycans having zero terminal galactose residues in a population of said antibodies is less than about 60%.

41. A method for treating a disorder in a mammal, comprising administering to the mammal a therapeutically effective amount of the antibody of claim 37.

42. A pharmaceutical composition comprising a monoclonal antibody of claim 37.

43. The pharmaceutical composition 42, wherein the monoclonal antibody is selected from the group consisting of an anti-HER2, antibody 2C4, anti-VEGF, antibody C2B8, anti-CD11a, anti-tissue factor, IgG4b, anti-CD40, anti-CD20, anti-IgE, antibody E25, and antibody E26.

44. A method of growing animal cells in fed batch cell culture, comprising
(a) growing animal cells in a cell culture medium, and
(b) culturing the animal cells in the cell culture medium, wherein glucose is added at the beginning of said culturing or during said culturing to create a glucose concentration during said culturing of greater than 10 g/L.

45. The method of claim 44, further comprising between said growing and said culturing, expanding the animal cells to provide an inoculum, and adding the inoculum to the cell culture medium at the beginning of the culturing.

46. The method of claim 44, wherein at some point of said culturing, the glucose concentration in the medium is at least about 12 g/L up to about 40 g/L.

47. The method of claim 44, wherein at some point of said culturing, the glucose concentration in the medium is a concentration selected group the group consisting of at least about 12, at least about 13, at least about 14, at least about 15, at least about 16, at least about 17, at least about 18, at least about 19, or at least about 20 g/L.

48. The method of claim 44, wherein the total amount of glucose added at the beginning and during said culturing is from about 10 g/L to and including about 40 g/L.

49. The method of claim 44, wherein the total amount of glucose added at the beginning and during said culturing is from about 15 to and including about 30 g/L.

50. The method of claim 48, wherein glucose is added to the cell culture medium at least once during said culturing.

51. The method of claim 50, wherein the glucose is in a batch feed.

52. The method of claim 51, wherein the batch feed is added in at least twice during said culturing.

53. The method of claim 44, wherein at the start of said culturing, the cell culture medium has an osmolality of about 280 to about 380 mOsm and at some point of said culturing has an osmolality of about 400 to about 600 mOsm.

54. The method of claim 44, wherein at the start of said culturing, the cell culture medium has an osmolality of about 300 to and including about 350 mOsm and at some point of said culturing has an osmolality of about 420 to and including about 500 mOsm

55. The method of claim 44, wherein said culturing begins at least 3 hours after the beginning of said growing.

56. The method of claim 44, wherein said culturing begins at least 12 hours after the beginning of said growing.

57. The method of claim 44, wherein said culturing begins at a time from about 3 hours to and including about 192 hours after the beginning of said growing.

58. The method of claim 44, wherein glucose concentration is maintained in said culturing to be at least about 2 g/L.

59. The method of claim 44, wherein the concentration of glutamine in the medium in said culturing at most about 5 mM.

60. The method of claim 44, wherein no glutamine is added to the medium during said culturing.

61. The method of claim 44, wherein glutamate is added to the medium during said culturing such that the concentration of glutamate is from about 1 to and including about 10 mM.

62. The method of claim 44, wherein during said culturing the ratio of glutamate concentration to glutamine concentration in the cell culture medium is at least 2:0.5.

63. The method of claim 44, wherein a batch feed comprising glucose is added to the cell culture medium at between 3 and 120 hours after the start of said culturing

64. The method of claim 63, wherein a batch feed comprising glucose is added to the cell culture during between about 24 and 72 hours, and again between about 80 and 120 hours, after the start of said culturing.

65. The method of claim 44, where during said culturing, the temperature of the medium is reduced by at least 2 degrees C. from the temperature of the medium at the beginning of said culturing.

66. The method of claim 65, wherein the temperature is reduced from a temperature between about 35 to 39 degrees C., inclusive, at the beginning of said culturing to a temperature between about 31 to 35 degrees C., inclusive, for the remainder of said culturing.

67. The method of claim 65, wherein the temperature is reduced at a time after the beginning of said culturing, wherein the time is between about 12 to about 72 hours, inclusive.

68. The method of claim 65, wherein the temperature is reduced about 2, about 3, about 4, or about 5 degrees C., at a time selected from the group consisting of at least about 12 hours, at least about 24 hours, at least about 36 hours, at least about 48 hours, and at least about 56 hours, after the beginning of said culturing.

69. The method of claim 44, further comprising between said growing and said culturing adding an inoculum of about 0.2% PCV seeding density to the cell culture medium, and shifting the temperature at about 24 hours after beginning said culturing, wherein the temperature is reduced from about 37° C. to about 34° C.

70. The method of claim 44, further comprising between said growing and said culturing adding an inoculum of about 0.4% PCV seeding density to the cell culture medium, and shifting the temperature at about 24 hours after the beginning of said culturing, wherein the temperature is reduced from about 37° C. to about 33° C.

71. The method of claim 44, further comprising between said growing and said culturing adding an inoculum having a seeding density of from about 0.1 to and including about 0.5% PCV.

72. The method of claim 44, wherein the cell culture medium is free of serum during the growing and culturing.

73. The method of claim 44, wherein the cell culture medium is free of animal-derived proteins during the growing and culturing.

74. The method of claim 44, wherein the cells are mammalian cells.

75. The method of claim 44, wherein the cells are Chinese Hamster Ovary cells.

76. The method of claim 44, wherein no butyrate is added during said culturing, and the culture medium is maintained at a pH between about 6.5 and about 7.5, inclusive, during said culturing.

77. The method of claim 44, wherein during said growing, the culture medium comprises about 1 to and including about 12 g/L of glucose, and wherein during or at the beginning of said culturing, a total of greater than 10 g/L of glucose is added to the culture medium.
